(19) United States
(12) Patent Application Publication
(10) Pub. No.: US 2010/0233241 A1
(43) Pub. Date: Sep. 16, 2010

(54) OPHTHALMIC DRUG DELIVERY SYSTEM AND APPLICATIONS

(75) Inventors: Charles Leahy, Concord, MA (US); Edward Ellis, Lynnfield, MA (US); Jeanne Y. Ellis, Lynnfield, MA (US)

Correspondence Address: Leason Ellis LLP, 81 Main Street, Suite 503, White Plains, NY, 10601, US

(73) Assignee: VISTA SCIENTIFIC LLC, Andover, MA (US)

(21) Appl. No.: 12/722,713

(22) Filed: Mar. 12, 2010

Related U.S. Application Data
(60) Provisional application No. 61/222,144, filed on Jul. 1, 2009.
(60) Provisional application No. 61/221,387, filed on Jun. 29, 2009.
(60) Provisional application No. 61/170,640, filed on Apr. 19, 2009.
(60) Provisional application No. 61/169,368, filed on Apr. 15, 2009.
(60) Provisional application No. 61/160,119, filed on Mar. 13, 2009.

(51) Int. Cl. A61F 2/00; A61K 38/13; A61K 31/5377; A61K 31/496; A61P 27/06; A61P 27/02

(52) U.S. Cl. 424/428; 424/427; 514/11; 514/236.2; 514/253.08

(57) ABSTRACT

An ocular device for insertion into an eye is provided and includes a body having an anterior surface and a posterior surface for placement on one of superior sclera and inferior sclera of the eye. The posterior surface is defined by a base curve that is substantially identical to a radius of curvature of the one of the superior sclera and inferior sclera of the eye. In one embodiment, the ocular device serves as an ocular drug delivery system and contains an active pharmaceutical agent, a lubricant, etc. In a second embodiment the ocular device can be constructed in such a manner to treat a wide variety of ocular conditions and diseases.

### CROSS-REFERENCE TO RELATED APPLICATIONS

The present application claims the benefit of U.S. patent application Ser. No. 61/222,144, filed Jul. 1, 2009; U.S. Patent Application Ser. No. 61/221,387, filed Jun. 29, 2009; U.S. Patent Application Ser. No. 61/170,640, filed Apr. 19, 2009; U.S. Patent Application Ser. No. 61/169,368 filed Apr. 15, 2009 and U.S. Patent Application Ser. No. 61/116,119, filed Mar. 13, 2009 which are hereby incorporated by reference in their entirety.

### STATEMENT REGARDING FEDERAL SPONSORSHIP

The U.S. Government has a paid-up license in this invention and the right in limited circumstances to require the patent owner to license others on reasonable terms as provided for by the terms of Grant No. 2 R44 EY13479-02 awarded by the National Institute of Health.

### BACKGROUND OF THE INVENTION

Due to the blood-aqueous and blood-retina barriers, it is difficult to get medicines administrated via the systemic route into the eye itself. Doses large enough to overcome these barriers often result in unacceptable systemic side effects. Virtually all acute and chronic disease of the eye are therefore treated with medication in the form of topical eye drop formulations that are applied at least once per day.

In addition to being difficult for patients to insert accurately, the use of eye drops suffers from two major technical disadvantages, their rapid elimination from the eye and their poor bioavailability to the target tissues. As a result of tear film dilution and elimination and the permeability barriers of the cornea, typically less than five percent of the applied dose of drug reaches the intraocular tissues. Topical ophthalmic pharmaceutical solutions are therefore formulated in high concentrations and require frequent dosing. Non-compliance with treatment, due to required frequency of dosing, lack of detectable symptom relief in immediate association with treatment application, undesirable systemic side effects due to the need for high concentrations of drug and other reasons, is a major clinical disadvantage.

The idea of placing a solid device into or near the eye to deliver a drug or a lubricant over time is not new. Most recent scientific interest in this field stems from advances in surgical techniques, pharmacology and pharmacokinetics, as well as the of improved polymer systems that can be tailored to the specific needs of ocular drug delivery. For clarity, the distinction should be made between a device that is “inserted into the eye”, meaning placed under the eyelids, external to the eyeball itself, and traditionally referred to as an “ocular insert”, vs. a device that is inserted into the eye surgically, meaning an intraocular insert placed inside the eyeball, or partly inside the eyeball itself. In fact, some devices are implanted in the layers of connective tissue forming the globe of the eyeball, and may even extend through these layers into the eyeball. And some that could be inserted topically under the eyelids could also be surgically implanted under the outermost layer, the conjunctiva, anteriorly, or Tenon's capsule, posteriorly, and would correctly be referred to as subconjunctival or sub-Tenon's inserts. This would be done via a minimally invasive procedure that does not open into the eyeball itself, but rather into the space currently utilized by ophthalmologists for subconjunctival or sub-Tenon's injections.

Ophthalmic inserts offer the following potential advantages: (1) increased ocular permanence with respect to standard vehicles, hence a prolonged treatment activity and a higher drug bioavailability; (2) accurate dosing (all of the drug is theoretically retained at the absorption site); (3) possible reduction of systemic absorption, which occurs freely with standard eye drops via the nasal mucosa; (4) better patient compliance resulting from a reduced frequency of medication and a lower incidence of visual and systemic side effects; (5) possibility of targeting internal ocular tissues through non-corneal (conjunctival-scleral) penetration routes; (6) increased shelf life with respect to eye drops, due to the absence of water; and (7) possibility of providing a constant rate of drug release.

Prior art has concerned itself with fitting a device under the eyelid into the conjunctival potential space. The goal to date has been to retain the device in this potential space, or potential pocket, formed by the palpebral portion of the conjunctiva (lining the inside of the eyelid) and the bulbar portion of the conjunctiva (lining the outside of the front half of the eyeball). The deeper parts of this potential pocket are the loose folds of the conjunctiva referred to as the conjunctival fornix or conjunctival cul-de-sac. This potential pocket of continuos tissue is limited by the eyelid margins, near the eyelashes, and the corneal limbus, the circle forming the border of the cornea with the white of the eye. It is referred to as potential space because it not particularly “designed” to hold anything normally, but rather the excess tissue allows movement of the eyeball in the orbit and retains foreign bodies and the tear film from going behind the eyeball into the head or brain. Being a soft, mucus membrane tissue, the conjunctiva easily swells in response to allergens or infection. The space it occupies is therefore potentially expandable by its outward pressure on the eyelids.

Devices meant to be inserted into this potential space have many shapes and sizes, and are often designed from the engineering standpoint of ease of manufacture. Resulting shapes are simple, such as oblong rectangular, cylindrical, etc. Their sizes and shapes are predicated on the art of tablet manufacture and the desire to be inconspicuous in situ. That is, comfort and retention in the conjunctival sac is attained by slipping something into the pocket formed by the conjunctiva lining the eyeball and the inside of the eyelid, and presuming it would be tolerated by the subject by virtue of its small size. This lack of design specific to the limiting contours of the intended space leads to discomfort and ejection of devices of any significant volume. This limitation of overall dimensions in turn significantly restricts the amount of drug they are able to contain and consequently deliver. An example of a commercially produced ocular insert for drug delivery is found in the subject of U.S. Pat. No. 3,618,604. This product was designed from an engineering standpoint of making a drug-releasing “sandwich”. Adequate retention and comfort were assumed by virtue of its small size. Several subsequent patents (U.S. Pat. Nos. 3,416,530, 3,828,777) also describe devices that are designed to improve drug delivery kinetics based primarily on material characteristics. These patents address design only in that the devices are adapted for insertion in the cul-de-sac of the conjunctiva between the sclera of the eyeball and the lower lid, to be held in place against the eyeball by the pressure of the lid. Although they are in fact quite small in comparison to the present invention, significant problems in retention and irritation occur with the use of these types of devices. In fact, the products have recently been discontinued, having never been widely accepted or used clinically.

Another example of prior art that utilizes the potential space of the conjunctival cul-de-sac is U.S. Pat. No. 6,217,896 (Benjamin). The '896 patent notes the failure to do so in the prior art, proposes to maximize the use of the actual volume and shape that could be contained in the cul-de-sac, addressing improved conformity, larger drug capacity and increased stability within the sacs. Benjamin's design is a result of maximally filling the potential space of the conjunctival cul-de-sac with a molding material, and describing the resulting shape obtained. Although his design description includes a back curvature conforming somewhat to the bulbar surface, this results from his approach of maximizing the volume and shape that could be contained in the human conjunctival sac. The features that he describes as unique to his design are those of the dimensions and volume of the expanded sac itself: “a crescent shape horizontally; a thick inferior horizontal ridge and a wedge-like shape sagittally”. The lack of well-defined mathematical dimensions or expressions for the design, or even a consistent recommended relationship between the back curvature and the bulbar surface, confirm his approach of molding the potential space by expanding it with molding material. As with other prior art, his invention is not designed to fit the eyeball itself and fits the potential space as an empirically derived molded design. Pulling the eyelid away from the globe would result in the insert sliding out of correct position or orientation and/or falling out of the eye.

Another example of prior art that includes a back curvature conforming to the bulbar surface also pursues the engineering approach of fitting a device into the potential space under the eyelid rather than fitting the eyeball itself. In U.S. Pat. No. 3,416,530, Ness describes an “Eyeball Medication Dispensing Tablet”. The hollow chamber of this patent is quite small, in order to comfortably fit in the cul-de-sac.

Much of the prior art depends on material flexibility to achieve retention, without specifying the material of the device or any values or ranges for the flexibility claimed. In WO 01/32140 A1 to Darougar, flexibility is claimed in claim 1 as being sufficient to allow bending along the curvature of the eye within the upper or lower fornix upon being positioned, such that the device does not extend onto any visible portion of the eyeball. The flexibility of Darougar et al is intended to allow entrapment of a long, thin device in the conjunctival folds of the fornix, and specifically excludes contact with the eyeball. The scope of the design of our invention allows incorporation of materials of any flexibility.

It is important to note that, other than Benjamin in U.S. Pat. No. 6,217,896, the history of the art of ocular inserts for drug delivery has been one of creating small devices, designed to be inconspicuous to the wearer while being trapped in the folds of the conjunctiva or between the eyelid and the globe. This has been addressed primarily by virtue of small size, and secondarily by virtue of shape. Special design features for stability consist of anchors to assist in entrapment, such as the protrusions mentioned in some prior art, such as WO 01/32140 A1 to Darougar, where the protrusions are quite small and are proposed as anchors to assist in entrapment of a long, thin rod-shaped device and render it undetectable in the conjunctival folds of the fornix.

Examples of prior art of considerably small volumes include the Ocusert® described above and the subject of U.S. Pat. No. 3,828,777, which measures at most 5.7.times.13.4 mm on its axes and 0.5 mm in thickness, yielding 38.5 μm volume. EPA-0262893 to Darougar discloses a rod-like ocular insert device having a volume of 17 μm. These restrictions on volume significantly limit the amount and subsequent duration of practical drug delivery to the eye.

When reviewing the prior art it is evident that the need exists for an ocular device that is both stable and comfortable in the eye, yet has the volume and mass to deliver therapeutic agents at a controlled rate over an extended period of time.

### SUMMARY

The present invention in a first aspect provides an ocular device adapted for the controlled sustained release of a therapeutic agent upon application onto the upper or lower sclera of the eye, said device designed to fit the sclera of the eye. The ocular device comprises an elongated body of a polymeric material said body containing a pharmaceutically active ingredient or a lubricant. The ocular device is fitted to the scleral curvature within the upper or lower fornix, upon being positioned so that the longitudinal axis of said device is generally parallel to the transverse diameter of the eyeball, said device being of a size and configuration such that, upon insertion into the upper or lower conjunctival area the device does not extend onto any normally visible portion of the eyeball, i.e., the palpepral aperture. The posterior surface of the device corresponds in a prescribed manner to the shape of the sclera, in a manner similar to how the posterior surface of a corneal contact lens corresponds in a prescribed manner to the shape of the cornea. The posterior edge of the ocular device can be tapered with a radius and a degree of edge lift in a manner similar to the edges of a corneal contact lens. The anterior surface can be designed to interact with the eyelid shape, tension and movement as the device occupies the anatomical potential space beneath the eyelid, in order to provide appropriate positioning, stability, movement and comfort.

The ocular devices of this invention have been designed to be stable in the eye and therefore well retained over a prolonged period of time. Additionally, the ocular devices are also designed to provide the patient with levels of comfort and tolerance not achieved with ocular inserts. The increased comfort, stability and retention of the ocular devices, fitted in the upper or lower conjunctival areas, can be used to deliver therapeutic agents to the eyes via continuous treatment for extended periods of time. One application of the device could be used for the singular or periodic treatment or prevention of inflammation, infection or allergy. Repeated applications for up to one to three months or longer each can be used for chronic diseases, such as glaucoma. The device may be fitted and removed by the ophthalmic technician, nurse or doctor, as well as by the patients themselves, following a brief lesson similar to that utilized for contact lens wear.

The ocular device is designed to be placed on the upper or lower conjunctiva, well within the junction of the palpebral conjunctiva of the upper or lower eyelid and the bulbar conjunctiva covering the sclera of the eyeball. Relative to the bulbar conjunctiva, the devices of this invention maintain their orientation, and exhibit only minimal movement vertically or laterally, by the pressure and movement of the eyelid against the eyeball, or by the movement of the eyeball itself. Slight movement of the device with blinking and eye movement is advantageous, as with contact lenses, to prevent adherence of the device to the eye and the associated entrapment of metabolic debris and deposits. Such movement relevant to the eyeball of a corneal contact lens is often referred to as “lag”.

The device may include raised areas, acting in use to maintain position and stability and minimize random movement of the device within the conjunctival area, preferably two raised areas each positioned so as to be symmetrically disposed about the center point of the body of the device.

The ocular device of this invention is designed to fit the sclera of the eye, which has a radius of about 11 mm to about 13 mm. Surprisingly, this radius in the adult population is relatively constant at about 12 mm. Therefore, the device has an overall, base curve radius of from about 11 mm to about 16 mm. Preferably, the device base curve radius is 12 to 14 mm.

In general, for adults, the area of the sclera limited by the upper fornix is greater than the area of the sclera limited by the lower fornix. Thus, an ocular device of the present invention with a length of up to 35 mm may remain on the upper sclera and one with a length of up to 25 mm may remain on the lower sclera without causing discomfort.

The length of the device of this invention is conveniently from 8 to 35 mm for use on the superior sclera to suit the eyes of different sizes such as infants, children and adults, or from 8 to 25 mm for use on the inferior sclera to suit the eyes of different sizes such as infants, children and adults.

The width (height of the vertical meridian with the device on the eye) of the device of this invention is preferably from about 1.0 mm to 14.0 mm to suit the eyes of different sizes such as those of infants, children and adults.

The edge of the device of this invention is preferably tapered and more preferably includes elements of the anterior and posterior peripheral surface, such as peripheral curve widths and radii and a resultant edge lift and an edge apex contour to optimize comfort and eyelid interaction.

The volume of the device of this invention can range from about 70 microliters to about 400 microliters and is preferably from about 100 microliters to about 200 microliters for adults. Infants and children under age five may require a device with a volume below 100 microliters.

The devices of this invention are well suited for various ocular applications for a controlled topical drug or agent delivery to the eye for enhanced treatment of a disease or condition. These applications are, but not limited, to the following: Glaucoma, Allergy, Infection (Bacterial, Fungal, and Virus), Inflammation, Post-surgical prophylaxis, Pain, Trauma, Dry eye, AMD, Diabetic macular edema, Uveitis, and Retinitis

The present invention can be utilized with various drugs and agents to be delivered to the eye, in a controlled manner, for the enhanced treatment of a disease or condition. It should be noted that the term “drugs and agents”, for the purpose of this invention, may also be expressed collectively as “therapeutic agents”.

There are a wide variety of drugs and agents available to treat the various aforementioned ocular diseases and conditions. While too numerous to list here it should be noted that any suitable ocular drug or agent, for a particular application, can be administered in a controlled manner in accordance with the practice of this invention. It should also be noted that combinations of drugs and/or agents can also be delivered to the eye in a controlled manner in the practice of this invention.

Suitable drugs or active agents that can be utilized with the present delivery devices include, by way of example only, but are not limited to:
Anti-infectives: such as antibiotics, including tetracycline, chlortetracycline, bacitracin, neomycin, polymyxin B, gramicidin, oxytetracycline, chloramphenicol, and erythromycin; sulfonamides, including sulfacetamide, sulfamethizole, sulfisoxazole; quinolones, including ofloxacin, norfloxacin, ciprofloxacin, sporfloxacin; aminoglycosides, including amikacin, tobramycin, gentamicin; cephalosporins; combinations of antibiotics; antivirals, including idoxuridine, trifluridine, vidarabine cidofovir, foscarnet sodium, ganciclovir sodium and acyclovir; antifungals such as amphotericin B, nystatin, flucytosine, fluconazole, natamycin, miconazole and ketoconazole; and other anti-infectives including nitrofurazone and sodium propionate.
Antiallergenics: such as antzoline, methapyriline, chlorpheniramine, pyrilamine and prophenpyridamine, emedastine, ketorolac, levocabastin, lodoxamide, loteprednol, naphazoline/antazoline, naphazoline/pheniramine, olopatadine and cromolyn sodium.
Anti-inflammatories: such as hydrocortisone, hydrocortisone acetate, dexamethasone, dexamethasone 21-phosphate, fluocinolone, medrysone, prednisolone, prednisolone 21-phosphate, prednisolone acetate, fluorometholone, fluorometholone acetate, meddrysone, loteprednol etabonate, rimexolone.
Nonsteroidal anti-inflammatories: such as flurbiprofen, suprofen, diclofenac, indomethacin, ketoprofen, and ketorolac.
Decongestants: such as phenylephrine, naphazoline, oxymetazoline, and tetrahydrazoline.
Miotics and anticholinesterases: such as pilocarpine, eserine talicylate, carbachol, diisopropyl fluorophosphate, phospholine iodide, and demecarium bromide.
Mydriatics: such as atropine sulfate, cyclopentolate; homatropine, scopolamine, tropicamide, eucatropine, and hydroxyamphetamine.

Furthermore, the following active agents are also useful in the present devices:
Antiglaucoma agents: such as adrenergics, including epinephrine and dipivefrin, epinephryl borate; β-adrenergic blocking agents, including levobunolol, betaxolol, metipranolol, timolol, carteolol; α-adrenergic agonists, including apraclonidine, clonidine, brimonidine; parasympathomimetics, including pilocarpine, carbachol; cholinesterase inhibitors, including isoflurophate, demecarium bromide, echothiephate iodide; carbonic anhydrase inhibitors, including dichlorophenamide acetazolamide, methazolamide, dorzolamide, brinzolamide, dichlorphenamide; prostaglandins, including latanoprost, travatan, bimatoprost; diconosoids and combinations of the above, such as a β-adrenergic blocking agent with a carbonic anhydrase inhibitor.
Anticataract drugs: such as aldose reductase inhibitors including tolerestat, statol, sorbinil; antioxidants, including ascorbic acid, vitamin E; nutritional supplements, including glutathione and zinc.
Lubricants: such as glycerin, propylene glycol, polyethylene glycol and polyglycerins.

The drug containing devices of this invention can be constructed to release the contained drug or agent by a variety of mechanisms for the controlled administration of a topical drug or agent to the eye for enhanced treatment of a disease or condition. These mechanisms include:

Physical or physiochemical systems

Chemical or biochemical

A combination of the above two systems

The physical or physiochemical systems include reservoir systems, matrix or monolithic systems, swelling-controlled systems or hydrogels, and osmotic systems or osmotic pumps or a combination of these processes

The chemical or biochemical systems are biodegradable polymeric compositions that can be degraded at the site of installation. The degradation of the polymer may be through hydrolysis, enzyme attack or microorganism breakdown, or a combination of these processes.

The devices of the present invention are constructed of polymeric materials or combinations of polymeric materials. The polymer matrix is chosen or formulated to optimize the release properties of the included drug or agents. In this manner the level of drug or agent in the device and the release profile can be engineered to provide effective treatment of the target disease or condition.

### BRIEF DESCRIPTION OF THE DRAWING FIGURES

Certain preferred embodiments and modifications thereof will become apparent to those skilled in the art from the detailed description herein having reference to the figures that follow, of which:

FIG. 1 is a diagrammatic sectional view of an eye and eyelid;

FIG. 2 is a front elevation view of an ocular drug delivery device according to a first embodiment;

FIG. 3 is a cross-sectional view taken along the line 3-3 of FIG. 2;

FIG. 4 is a perspective view of an eye with the device of FIG. 1 fitted to the superior sclera;

FIG. 5 is a perspective view of an eye with the device of FIG. 1 fitted to the inferior sclera;

FIG. 6 is a front elevation view of an ocular drug delivery device according to a second embodiment;

FIG. 7 is a cross-sectional view taken along the line 7-7 of FIG. 6;

FIG. 8 is a cross-sectional view taken along the line 8-8 of FIG. 6;

FIG. 9 is a front elevation view of an ocular drug delivery device according to a third embodiment;

FIG. 10 is a top plan view of the device of FIG. 9;

FIG. 11 is a cross-sectional view taken along the line 11-11 of FIG. 9;

FIG. 12 is a cross-sectional view taken along the line 12-12 of FIG. 9;

FIG. 13 is a front elevation view of an ocular drug delivery device according to a fourth embodiment;

FIG. 14 is a cross-sectional view taken along the line 14-14 of FIG. 13;

FIG. 15 is a cross-sectional view taken along the line 15-15 of FIG. 13;

FIG. 16 is a plot of cumulative weight, in micrograms, of Timolol drug released versus time; and

FIG. 17 is a plot of cumulative weight, in micrograms, of Ciprofloxacin drug released versus time.

### DETAILED DESCRIPTION OF PREFERRED EMBODIMENTS

The present invention incorporates principles that have some basis in rigid gas permeable and soft corneal contact lens design and more particularly, the engineering of ocular devices, according to the present invention, is particularly suited for producing devices for drug delivery to the eye while being fitted to the sclera (white) of the eye. Accordingly and as described in great detail below, the device designs described herein address a back central curvature, peripheral curves, edge apex contour, edge lift, overall shape and thickness profile corresponding to the features of and delimiting aspects of the superior and inferior sclera, such as the scleral surface curvature, extraocular muscle insertion points, corneo-scleral junction contour, and the corresponding eyelid interaction. In complete contrast to prior art devices and drug delivery approaches, the present ocular devices are specifically designed to fit the sclera of the eye, with the overall fitting contour accounting for the limiting anatomical features and landmarks of the sclera, such as the extraocular muscle insertions and limbal junction with the cornea. The devices are held in place by fluid attraction, and the devices interact with the eyelids, as does a contact lens, for movement, positioning, stability and comfort. The posterior contour allows comfortable relative apposition to the scleral surface, and allows movement with blinking and eye movement. The anterior contour, edge design and the thickness profile of the embodiments of this invention interact with the eyelid both during and between blinks to optimally orient the device in a stable and comfortable position on the sclera. Each device is inserted by placing it on the inferior or superior anterior sclera (white) of the human eye or in treatment of primates and quadrupeds, as a contact lens is typically placed on the clear cornea. The design of the device does not require insertion into the conjunctival cul-de-sac for retention. The design allows the device to remain in place even if the eyelid is retracted, just as a contact lens remains in place when the eye is open. This design can be utilized in its embodiments with a wide range of drugs, lubricants and other medicinal agents, and with a wide range of potential eroding and non-eroding drug delivery materials or combinations of materials, such as via polymer matrix chemistry or reservoir systems. The polymeric material of the device may be any polymer that is above its gas transition at 35° C. For example, a silicone elastomer, acrylate, and methacrylate compositions and hydrogels are suitable. The mechanisms of the therapeutic agent or lubricant release may be, for example, by diffusion through the matrix of the device, by diffusion through an outer wall of the device, osmosis and bioerosion. The design of the device allows large volumes of drug to be delivered over a long duration.

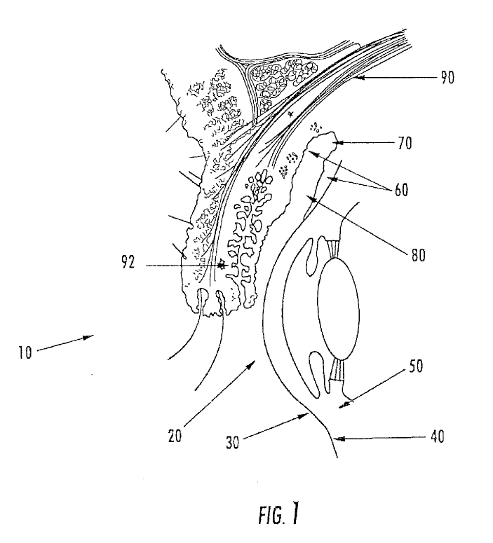

With reference to FIG. 1, the following definitions and terms may be useful regarding the anatomy of the anterior eyeball and the description of the details of the invention. When describing the eye, it is convention to describe it by using a number of different established anatomical terms. FIG. 1 shows an eye 10 that includes a cornea 20 which is the transparent anterior portion of the eyeball and has a steeper curvature than the rest of the eyeball. The corneal limbus 30 describes an annular border zone between the cornea 20 and the bulbar conjunctiva 40 and the sclera 50. The conjunctiva 60 refers to the mucous membrane extending from an eyelid margin to the corneal limbus 30, forming the inner layer of the eyelids and an anterior outer layer of the eyeball. The conjunctival fornix 70 is the loose, free conjunctiva connecting the eyelid (palpebral) and eyeball (bulbar) portions of the conjunctival cul-de-sac 80 which is the potential space between the bulbar and palpebral conjunctivae and in the conjunctival fornix that can expand into a real space by insertion of a device or other object or substance. The palpebral conjunctivae are supported by the various muscles 90 and embedded glands 92 of the eyelid. As previously mentioned, the sclera 50 is the white, opaque outer tunic of the eyeball which covers it entirely except for the segment covered anteriorly by the cornea 20. The sclera 50 is in turn covered anteriorly by the conjunctiva 60.

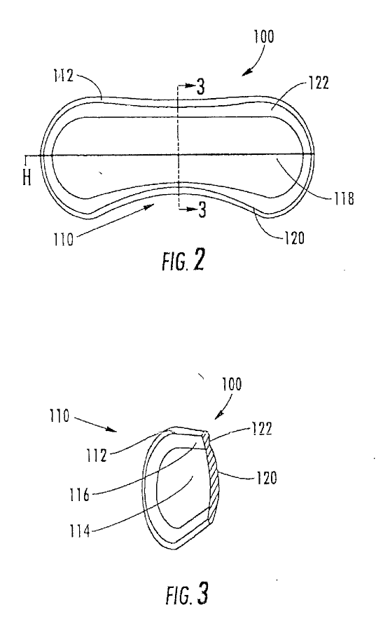

With reference to FIGS. 1-3, FIGS. 2 and 3 generally illustrate an ocular drug delivery device 100 that embodies the features of the present invention and is constructed for insertion into and wear in the eye 10 by placing it on the inferior or superior anterior sclera (white) 50 of the human eye 10 or in treatment of primates and quadrupeds. The device 100 is initially set forth in FIG. 2 in order to define a number of design terms that help describe the structure and function of all of the present ocular drug delivery devices. Thus, it will be understood and will become more apparent below that the device 100 is merely one exemplary embodiment of the present invention and in no way is to be construed as limiting the scope of the present invention.

The device 100 includes a body 110 that has an edge apex contour 112 which is the amount and positioning of rounding of the device edges and is typically defined as a radius profile swept around a perimeter of the device 100. The device 100 has a base curve 114 which is defined as the primary radius in each meridian i.e. vertical (axis 3-3) and horizontal (axis H-H), and is the surface of the device 100 that is in contact with the sclera 50 (the posterior surface of the device). In the case where the values in each meridian are the same, the base curve 114 is defined as a spherical base curve. In the case where the values in each meridian are different, the posterior surface is defined as a tone posterior surface. The device 100 also has an edge lift 116 which is a sectional geometry width around the perimeter adjacent to and following the edge apex contour 112 where the base curve 114 is flatter (increased). The edge lift 116 is defined by the incremental radius increase and by a width.

A front curve(s) 118 is defined as the secondary device radius in each meridian i.e. vertical and horizontal (axes defined along the body 110). The front curves generate the surface that is in contact with the lid (the front surface of the device). In the case where the values in each meridian are the same, the front curve 118 is defined as a spherical. In the case where the values in each meridian are different, the front surface of the device 100 is defined as a toric front surface. In a preferred embodiment, the present device 100 disclosed herein, the front curves 118 are defined as tonic. The device 100 also includes splines 120 which are geometric entities created by polynomial equations, which define smooth blended contour surfaces bridging from one defined shape or cross-section to another. A lenticular 122 is a manipulation of the thickness of the edge of the device 100 at the front curve geometry adjacent to the edge apex contour 112 on the eyelid side of the device 100. A lenticular 122 can be a positive or a negative curve and typically has a reversed radius direction to the primary front curve radius geometry and the lenticular 122 follows the profile of the edge apex contour 112, thus providing a reduced thickness cross-section profile around the perimeter of the device 100.

The body 110 of the device is constructed and configured to fit the contours of the white part (sclera 50) of the eyeball itself, while paying tribute to the effects of the eyelids on the position, stability, movement and comfort of the device 100. This fit can be analogized to the design and fitting of a corneal contact lens over the contours of the cornea 20. While the primary function of the contact lens is to optically correct a refractive error, the lens must also be designed to be comfortable, stable and non-irritating, and to remain in place in order to function successfully. Although remaining in place, it also must retain a slight movement with eyelid movement and a slight lag behind movement of the eyeball. This is to permit tear film circulation around the lens to prevent redness, irritation, adherence to the tissue and build-up of mucus and other surface deposits on the anterior or posterior surfaces. Similarly, an ocular device, such as device 100, for drug delivery also must exhibit stability of position and yet would preferably retain slight movement and lag for the same reasons. It also cannot cause excessive awareness or create discomfort as wearing time proceeds. The interaction with the lid is also determined by the design, and, as with a contact lens, will affect the position, stability, movement and comfort of the device 100. Proper interaction of the device 100 with the eyelid also allows flow of the tear film around the device 100, which helps keep it clean of mucous build-up that tends to occur with foreign bodies that are simply trapped in the conjunctival cul-de-sac 80.

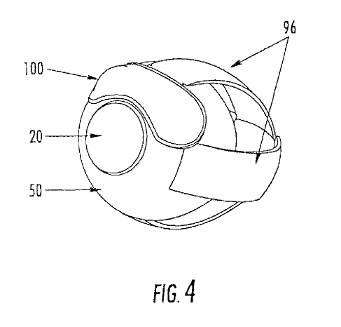

The device 100 of this invention can be worn over the sclera 50 superior to the cornea 20 as shown in FIG. 4 or inferior to the cornea 20 as shown in FIG. 5. It will therefore be appreciated that all of the ocular drug delivery devices embodying the principals and features of the present invention can be positioned in either of these two locations and can be marked as such.

Contact lens fit and retention depends on the attraction of the device to the eye by the surface tension of the tears (fluid attraction), and is assisted by the curvature of the back of the contact lens. Typically a contact lens has a back curvature corresponding (according to relationships known to those in the art) to that of the cornea, so that the lens has a preference for being attracted to the surface of the cornea as opposed to the sclera, or white part of the eye. The general attraction of the contact lens to the eye is evidenced by the fact that a contact lens does not simply fall out if the wearer tilts the head down while the eyes are open.

The attraction of the contact lens to a specific part of the eye (the cornea 20) is evidenced by the observation that, with the eye wide open, the lens moves with the eye, such as left, right, up or down with change of gaze direction. This preferential attraction of the contact lens to a particular part (shape) of the eyeball, specifically, the more steeply curved cornea 20 vs. the more flat sclera 50, can be demonstrated if the eye is held open wide and a soft contact lens is dragged from the cornea 20 to the white part 50 of the eye, leaving only a small portion remaining over the cornea 20. The contact lens will drift back onto the cornea 20 on its own without a blink as long as the eye remains wet enough. This is because the contact lens is specifically designed, by the series of posterior base (central) and peripheral curves and the diameter, thickness, etc., to position in close relationship to the cornea 20. In sum, the design and intent of contact lens wearing is to position the contact lens over the cornea 20 and there is absolutely no teaching or suggestion of placement of the contact lens in another anatomical area of the eye 10. In fact, the contact lens is not suitable for placement in other areas, including the sclera 50 specifically.

Thus, contact lens design and wear is in complete contrast to the present invention, where the device 100 is designed to fit the contours and anatomical features of the white part 50 of the anterior eye, in order to remain in position on the sclera 50. Currently available contact lenses, although designed with several desirable attributes of ocular devices for drug delivery, such as adequate comfort, retention and movement, do not provide significant drug delivery capability. This is due to the inability of the lens materials to deliver drug for significantly long duration. Most studies investigating contact lenses pre-soaked in drug solutions show release of all of the drug in a matter of hours or perhaps one to two days. The constraints of the contact lens materials available having adequate optical clarity (for vision) and oxygen permeability (required for adequate metabolism in the avascular cornea) do not allow high priority in material choice of polymers that offer extended drug delivery. Thus, previous drug delivery design which focuses on mimicking a contact lens design suffers from a number of disadvantages.

The invention disclosed herein is specifically designed to fit the non-corneal (scleral) anterior surface of the eyeball, remaining outside the visual axis and off of the avascular cornea. Therefore, optical design, optical clarity and oxygen permeability are not constraining parameters to the materials that can be used with the design comprising this invention.

The device 100 is constructed to be retained at the non-corneal anterior ocular surface for the topical delivery of drug to the eye. Contrary to existing ocular drug delivery thought in terms of the mechanism of topical drug delivery, the present device 100 is specifically designed to fit the sclera 50 of the eye 10. This is evidenced by the fact that each embodiment of the present device 100 stays on the sclera 50 even if the eyelid is pulled away from the eye 10, similar to how a contact lens stays on the cornea 20 while the eye is wide open. This is a different approach than that of conventional ocular drug delivery design that relies on entrapment of the device in the folds of the conjunctival sac or between the eyelid and the globe for its retention in position.

However, along with retention, the term “fit” in the contact lens field also encompasses positioning, stability, movement, eyelid interaction and even comfort. As with contact lens designs, there are specific design features that render the device 100 described in this application capable of performing adequately in all these aspects of “fit”. Due to its design to fit the sclera 50 of the eye 10 and account for dynamic interaction with the movement of the eye 10 and of the eyelid, the present device 100 provides comfort in a large design. The total device volume can be much greater than device volume in much of the prior art, which is significantly limited by that size which creates detectable sensation or discomfort.

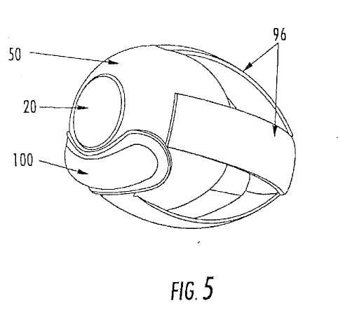

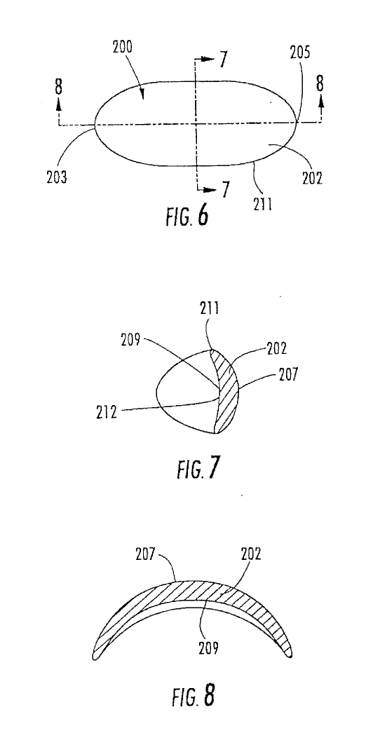

The ocular devices of this invention, in their simplest form, are designed to fit the sclera 50 of the eye. Generally, most of the devices include a body that has a generally overall oval shape where the horizontal dimension is greater than the vertical dimension. This is depicted in the embodiment shown in FIGS. 6-8, where an exemplary ocular device 200 is provided. The ocular device 200 has a body 202, a first end 203 and an opposing second end 205 as well as an anterior surface 207 and an opposing posterior surface 209 that are closest to one another along a peripheral edge 211 of the body 202.

It is preferred that the shape be symmetrical about a medial axis (vertical meridian) that extends across the width of the body 202 (e.g., line 7-7 of FIG. 6), such that the lateral halves are mirror images. This aspect allows for the same device design to be used in the right and left eyes (in the same orientation), and on the superior or inferior sclera 50 of eye 10. A base curve 212 radius of the device 200 is chosen to fit the sclera 50. As best shown in FIG. 7, the body 202 has a thickness that is less at its edges 211 and greater toward and including the middle of the body 202. More specifically, the body 202 can be designed such that it has a maximum thickness at the middle thereof as measured from each of the side edges of the body 202 and as a result, the maximum thickness generally lies along the line 8-8 (horizontal meridian) of FIG. 6. One will appreciate that as a result of this configuration, the thickness of the device 200 continually increases from each side edge toward the middle of the body 202.

In addition, the cross-sectional thickness of the body 202 from the first end 203 to the opposing second end 205 is likewise not uniform but instead tapers inwardly toward each end 203, 205 from the central section (middle) of the body 202, as best shown in FIG. 8. In terms of a maximum cross-sectional thickness of the body, as measured longitudinally from the first end 203 to the second end 205, this generally lies along the line 8-8 of FIG. 6. The body 202 thus tapers in the longitudinal direction from its central region toward the ends 203, 205 such that the distance between the anterior surface 207 and the posterior surface 209 is at a greatest in the central region, while is at a minimum at the ends 203, 205 and more particularly along the peripheral edge 211 of the body 202. The edge thickness, measured along the perimeter edge 211, of the body 202 is generally uniform along the entire perimeter of the elliptical body 202 where the anterior surface 207 and the posterior surface 209 meet. Accordingly, this body design is characterized as being a significant toric shape on a fairly spherical base curve with a uniform edge radius. In one exemplary embodiment the device 200 can have the following dimensions: the width can range from about 10 mm to about 25 mm, the height is about 5 mm to about 12 mm and the cross-sectional thickness (center thickness) is from about 1.0 mm to about 3.0 mm as measured through the center of the body 202, i.e., along line 7-7 of FIG. 6. The base curve radius of the device 200 is from about 12 mm to about 14 mm. When the device 200 has the above dimensions, the volume ranges from about 72 microliter to about 400 microliter. It will be appreciated that the aforementioned dimensions are merely exemplary in nature and do not serve to limit the present invention in any way since it is possible for the device 200 to have one or more dimensions that lie outside of one of the above ranges but still be completely operable as an ocular delivery device.

As previously mentioned, the present inventors discovered that the device 200 is particularly suited for and is in face constructed and configured for placement on the either the superior sclera as shown in FIG. 4 or the inferior sclera as shown in FIG. 5. Not only is the device 200 comfortable to wear in these locations but also it delivers the aforementioned advantageous drug delivery properties that were otherwise not achievable in conventional ocular devices that were inserted into the eye 10 and worn at locations other than the sclera 50, such as the cornea 20.

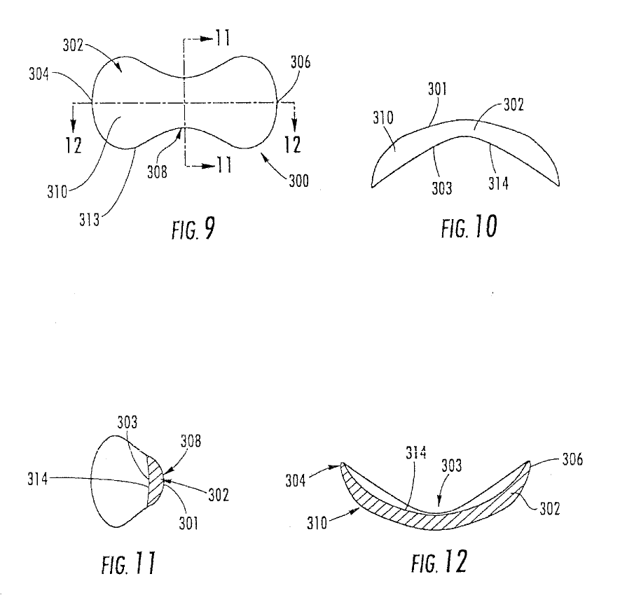

FIGS. 9-12 illustrate an ocular drug delivery device 300 according to a second embodiment of the present invention. The ocular drug delivery device 300 shares a number of similarities to the device 200, such as both being intended for placement on the sclera 50; however, there are a number of differences in terms of the construction and design of the device 300 compared to the device 200. Similar to the device 200, the device 300 has a degree of symmetry in that the device 300 has a body 302 that is preferably symmetric about a central axis that is defined as being equidistant from a first end 304 and an opposing second end 306 of the body 302 and extending between the two sides of the body 302. This central axis (vertical meridian) is depicted as line 11-11 in FIG. 9. As with the device 200, the device 300 includes an anterior surface 301 as well as a posterior surface 303.

As best seen in the front elevation view of FIG. 9, the device 300 generally takes the form of a “dumbbell” with a relatively thin central section 308 and two opposing lobe sections 310 formed at ends 304, 306, respectively. The central axis aspect ratio of the lobe 310 to the central section 308 (vertical meridian 11-11, as viewed from the front elevation view of FIG. 9) can vary from about 2:1 to about 10:1. In theory, the central portion 308 could be infinitely narrow and thin, but increasingly negative effects on stability and comfort would occur as such dimensions were approached and therefore, the above ranges, while not limiting, serve as a guideline for yielding a suitable device 300. The dumbbell shape of the device 300 redistributes the mass away from the center 308 towards the ends 304, 306 of the device 300, and leads to desired positioning on the sclera 50 under the lid and greater stability on the eye 10 while maintaining volume.

Increasing the mass in the periphery of the device 300 also takes advantage of greater scleral surface area available in the forty-five degree quadrants vs. the central axis (superior and inferior), which are limited by the extraocular muscle insertions (superior or inferior recti muscles). The larger shape of the lobes 310 relative to the central portion 308, the greater height of the lobes 310 from the surface of the eye and the surface contour of the lobes 310 all contribute to the proper positioning, stability and movement of the device 300 on the sclera 50. Although the lobes 310 can be of any geometrically shaped perimeter, for optimal interaction with the eyelid and the blink process, the perimeter of the lobes 310 distal to the central connecting portion 308 generally has a rounded appearance as viewed in the top plan view of FIG. 9, and can have parabolic shapes at the ends 304, 306 with splines between them.

The lobes 310 can be from about 0.5 mm to about 20 mm at their greatest diameter. More preferred is a diameter from about 3 mm to about 17 mm. Most preferably, the lobes 310 can be from about 7 mm to about 13 mm at their greatest diameter. The center thickness, as measured from the anterior surface 303 to the posterior surface 301 (similar to the same measurement in a contact lens) of the central portion 308 of the device 300 can range from about 0.50 mm to about 4.0 mm, more preferably from about 0.10 mm to about 2.0 mm, and most preferably from about 0.10 mm to about 1.25 mm, while a thickness, measured across a central section, of the lobe 310 can range from about 0.5 mm to about 5.0 mm, more preferably from about 0.5 mm to about 3.0 mm, to avoid visible bulging through the eyelid, and most preferably from about 0.5 mm to about 2.5 mm. The greater thickness and volume of the lobes 310 compared to other regions of the body 302 retains adequate volume for clinical quantities of drug delivery while maintaining position and stability on the eye through interaction with the eyelid. Keeping the thickness profile of the central portion 308 below that of the lobes 310 decreases the potential volume available, but offers significant benefits in position, stability, appearance (no bulge noted through eyelid) and comfort in the use of the device 300. The nasal and temporal perimeter (“ends”) 304, 306 of the lobes 310 can approximate circular, parabolic or elliptical shapes. The transitional curves between the central portion 308 of the device 300 and each of the lobes 310 can be linear, parabolic, elliptical or hyperbolic, with splines being preferred, blending to a central cross-section at line-line 12-12. The overall horizontal width of the device 300 can range from about 10 mm to about 25 mm, with a base curve radius 314 from about 12 mm to about 14 mm. The overall volume of the device 300 ranges from about 70 microliter to about 400 microliter. The thickness of the device 300 tapers down to a defined minimum, mostly uniform edge thickness around the entire edge perimeter 313.

The symmetry of the device 300 about the vertical meridian (axis 11-11 (vertical meridian)) is such that the lateral halves are mirror images. This aspect allows for the same device design to be used in the right and left eyes (in the same orientation) and on the superior or inferior sclera 50 of the eye 10.

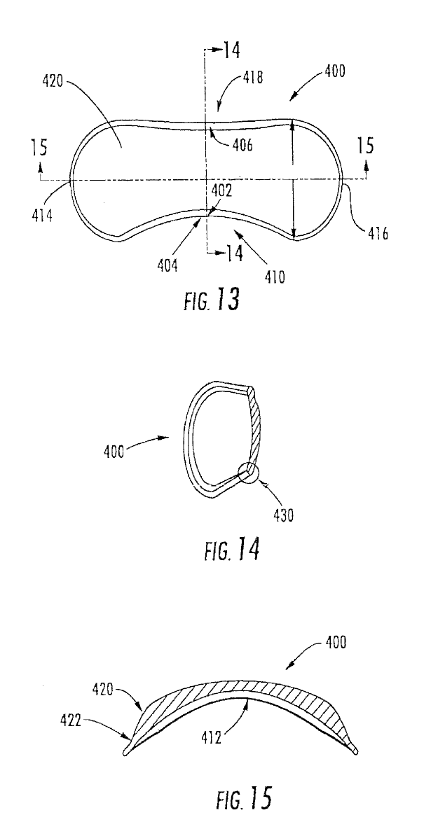

In yet another embodiment that is illustrated in FIGS. 13-15, an ocular drug delivery device 400 is provided. In a number of intended applications, the embodiment of device 400 is preferred over the other prior embodiments (devices 200 and 300) for the reasons set forth above. More specifically, the device 400 is designed to better fit the anatomical features of the eye 10. In this embodiment of the invention, an edge 402 of a central portion 404 thereof that is proximal to the cornea 20 during placement on the eye 10 has a shape corresponding approximately to a projection of the corneal perimeter. This inwardly curved shape has a curvature such that if you projected the corneal boundary (at the limbus) and the device 400 boundary into a corneal plane, the device 400 would have an approximately uniform clearance in relation to the corneal boundary when the device 400 is in its intended position on the superior or inferior sclera 50. This feature is termed the “corneal relief curve” and is generally indicated at 410. The curvature of the corneal relief curve in this design is a conic or spline projection of the curvature of the junction of the corneal and sclera (the limbus). Most preferably, it follows a uniform offset radially from the limbus along the sclera 50. The height difference, as measured parallel to 14-14, due to this inward curvature of the central axis 14-14 (vertical meridian) between the center of the device 400 and lobe portions 420 can range from about 0.50 mm to about 3.5 mm, and more preferably, from about 0.50 mm to about 2.5 mm. The “relief contour” provides a shape that will not impinge on the sensitive corneal surface, thereby avoiding effects on comfort and potentially vision, and approximates a uniform clearance in relation to the cornea 20.

The edge 406 of the central portion 404 distal to the cornea also has an inwardly curved shape, with a curvature allowing clearance of the insertion of the rectus muscle (superior or inferior, depending upon placement of the device on the superior or inferior sclera). This feature is termed a “muscle relief curve” and is generally indicated at 418. The height difference, due to this inward curvature, of the central axis 14-14 between the center of the device 400 and the lobe portions 420 can range from about 0.15 mm to about 2.5 mm, or more preferably, from about 0.15 mm to about 1.5 mm.

Symmetry about the center axis 14-14 (vertical meridian) in FIG. 13 is maintained in such an embodiment, allowing it to be worn inferiorly or superiorly in most cases, but the mass of the central portion 404 is greater on the side of the longitudinal meridian 15-15 of FIG. 13 that is distal to the cornea, so that in the superior position, the inward curvature 418 of the device 400 clears the superior rectus muscle insertion, but is less of an inward curvature than that 410 on the side proximal to the cornea.

The center thickness along line 14-14 (vertical meridian) varies from about 0.25 mm to about 3.0 mm according to one embodiment, a longitudinal length of the device 400 measured from end 414 to end 416 ranges from about 15 mm to about 22 mm, and the maximum vertical height (as viewed from the side elevation view of FIG. 14) ranges from about 5 mm to about 14 mm. The distance at the center point across this central portion 404, from proximal to distal relief curves, along the axis 14-14, can vary from less than about 0.5 mm to about 12 mm. More preferred is the range of from about 1 mm to about 10 mm. Most preferred is the range of from about 6 mm to about 10 mm. The centers of each dumbbell (each end lobes) 420 on either side of the central portion 404, can range in thickness from about 0.5 mm to about 5.0 mm, more preferably, from about 0.5 mm to about 3.0 mm, to avoid visible bulging through the eyelid, and more preferably, from about 0.5 mm to about 2.5 mm. The lobes 420 can contain the greater part of the volume of the device 400, which ranges from about 70 microliter to about 400 microliter.

The base curve radius, generally indicated at 412, of the device 400 ranges from about 12 mm to about 14 mm.

Each end lobe 420 has a mid-peripheral section 422 that is thinner than the peripheral portion of each end lobe 420. This is to mimic the edge profile technique typically used in the geometry of a significantly high powered rigid contact lens. Such high powered lenses have been observed to be most likely of common clinical corneal contact lens designs to dislocate from the cornea, due to the interaction with the superior eyelid. The volume of such a contact lens is necessary to provide adequate optics for visual correction. Similarly, the volume of the device 400 is necessary to provide adequate drug for release. In both cases, the lenticular feature is a benefit in maintaining position and stability, through interaction with the eyelid, of the device 400 that has sufficient volume. The lenticular feature yields a transition from a positive front apical curve of the lobe 420 being blended into a negative reverse curve in a range from about 0.5 mm to about 3.5 mm.

The symmetry of the device about the axis 14-14 (vertical meridian) is such that the lateral halves are mirror images. This aspect allows for the same device design to be used in the right and left eyes (in the same orientation) and on the superior or inferior sclera of an eye (by rotating 180 degrees in the corneal plane).

In all embodiments, the back surface approximates the primary scleral curvatures, at least in situ, depending on the flexibility of the material. The flexibility of the material utilized to form the device determines how closely the back surface must correspond to the scleral curvatures prior to insertion of the device. For example, in theory, a highly flexible material could be made with larger base curve radii, and could conform in use to form itself to the surface of the sclera. This is comparable to the “draping” effect of a soft contact lens on the eye.

The present invention utilizes conformation to the eyeball curvature to establish the fit against the surface of the eyeball, not to assist with entrapment in the conjunctival folds of the fornix. The design of this invention aims to provide a surface geometry to fit the sclera 50 of the eye 10 in order to balance comfort and retention with a greater volume of the device to contain greater amounts of drug for longer delivery to the eye. Adjusting the base curvature and peripheral curvatures of the posterior surface of this invention allows the use of many materials with a wide range of flexibility. Such adaptation of design to materials properties is well known in the art of contact lens design. A range of spherical, aspheric and tonic back surface base curves, in combination with various spherical, aspheric and tonic peripheral curve systems, similar to those known in the art of contact lens design, provide the posterior surface that fits against the surface of the eyeball.

Therefore, although a flat posterior surface is within the range of possible posterior surfaces of this invention, the preferred range of volumes of the device of this invention would result in less of a draping effect and a more limited tendency to conform to the scleral surface if the posterior surface were flat prior to insertion in the eye, virtually regardless of material utilized. This is comparable to a thick soft contact lens, such as a high plus power lens used for the correction of aphakia, draping, flexing or bending less on the eye than a very thin, low power soft contact lens. It can be noted analogously that even a thin low power soft contact lens, which is quite flexible, is manufactured with a base curvature corresponding somewhat to the ocular (corneal) curvature, as opposed to a flat posterior surface, to assist with fitting and draping. In a preferred embodiment of this invention therefore, the device would have a posterior surface approximating the scleral curvature.

In fact, the surface of the anterior sclera forms a somewhat tonic, asymmetric surface. This would be analogous to fitting a contact lens on the more asymmetrical mid-peripheral cornea, rather than basing the design on a central corneal topography. A back toric design posterior aspheric surface contact lens would be applicable for use on such a toric surface. A more preferred embodiment would therefore have a posterior surface with an aspheric shape or with two spherical radii that would allow it to conform to the scleral curvatures. Although potential drug delivery devices with a spherical back surface design would adequately approximate the scleral surface, the flattening and steepening of elliptical or aspheric back curvatures would allow fine tuning of the movement and tear flow, and hence the optimal fit of the device.

Another advantage of specific designs of the back surface of the device is to allow uniform tear film flow. More uniform tear flow would allow more constant release of the drug from the device to the eye. Therefore, although a tonic back surface is not necessary for the more flexible materials, it would be preferred for the positioning, stability, comfort, retention and uniform drug delivery with the more rigid materials. The most preferred embodiment of this invention therefore comprises a posterior surface with two elliptical radii that would allow it to conform to the slightly elliptical scleral surface. These elliptical radii can result from the manufacturing process or from the in situ conformation of a spherical radii device of flexible materials. The edge lift radii of the peripheral curves 430 can range from 0.0 to 5.0 mm flatter than the base curve radii in each meridian. More preferred is 0.50 to 5.0 mm flatter than the base curve radii in each meridian. Most preferred is from 2.0 to 5.0 mm flatter than the base curve radii in each meridian. The peripheral curve 430 widths can range from 0.10 to 2.0 mm. More preferred is 0.10 to 1.0 mm. Most preferred is from 0.25 to 0.75 mm. The resulting edge profile incorporates the peripheral curvatures 430 of the anterior surface and the posterior surface of the device 400.

A contact lens design utilizes lid interaction during the blink and/or interblink period to optimally position the contact in relation to the cornea. As with a contact lens design, the most preferred embodiments of this invention have critical design features of anterior shape, edge contour and thickness profile that interact with the eyelid, both during and between blinks, to optimally orient the device in a stable and comfortable position, in this case, on the sclera.

An example of such a design feature of this invention that is well known in the art of contact lens design is that of the addition of a minus-carrier lenticular. This design feature affects the edge profile thickness and affects the interaction with the eyelid. This is known to aid in comfort as well as to stabilize and position the contact lens in the desired position on the eye. In a similar manner, the lenticular designs of our more preferred embodiments position and stabilize the ocular devices in the optimal position on the sclera. In fact, it can be observed in the art of contact lens practice that a rigid corneal contact lens with a minus carrier lenticular, if dislocated onto the superior sclera accidentally, tends to want to remain stable in that position. This is in spite of the other design features of the lens that would tend to have it return to the cornea. This interaction of a minus-carrier lenticular-type peripheral profile with the eyelid has been utilized in the most preferred embodiment of the present invention to optimize the position and stability of the device either in the superior or inferior position on the sclera. The more preferred embodiments utilize a lenticular on the lobes that is of larger radius than that of the central portion of the device. The lenticular radius is therefore smallest at the central vertical meridian of the device, with the distal (non-corneal) side lenticular radius at that point being closer to the larger lenticular radius of the lobes and having a larger (approximately double the size) radius than that of the proximal (corneal) side. In the preferred embodiments of the invention the lenticular is carried all the way around the perimeter of the device to assist in maintaining location of the device by the lid, balance of position and movement of the device with blinking, and minimal awareness of the device or foreign body sensation with lid movement. The lenticular radii for the distal (non-corneal side) central vertical meridian, proximal (corneal side) central vertical meridian and lobe range respectively from: preferred 0.0-5.0, 0.0-5.0, 0.0-5.0 mm; more preferred 0.5-3.5, 0.5-3.5, 0.5-3.5 mm; most preferred 1.0-2.0, 0.25-1.5, 1.5-2.5 mm. The lenticular enhances balance and minimizes sensation of the device in interaction with the lid contact area. Stability and retention in the face of movement of the superior lid is particularly optimized with the use of a lenticular design.

The same elements of design resulting in the overall shape and surfaces and edge geometry of the embodiments of this invention allow the surgical placement of the device of this invention under the conjunctiva or Tenon's capsule for delivery of drug to the anterior or posterior of the eye 10. The overall shape of the preferred embodiments would fit into position anterior or posterior to a given extraocular muscle insertion. In the case of being placed posterior to a muscle insertion, the muscle relief curve would maintain its function, while the corneal relief curve would become an “optic nerve” relief curve. Primarily due to the curvatures on the anterior and posterior surfaces and the edge apex contour, there would be minimal structural interference with the tissues surrounding the device of this invention, during surgical insertion, wear and surgical removal, if necessary. The maximized volume of the device as described in each of the present embodiments allows delivery of significant quantities of drug in order to minimize the number of surgical replacements necessary, yet remain unobtrusive in the normal movements and sensations of the eye.

The present invention describes the design of an ocular device that overcomes the deficiencies associated with the conventionally designed ocular devices and incorporates one or more of the following features: (a) the ocular device is designed to fit the sclera of the eye; (2) the ocular device is designed to be retained on the eye independent of the eyelid; (3) the ocular device is designed to move and position with the blink; (4) the ocular device is designed such that the base curvature of the device is spherical, aspherical, or tonic and is defined in relation to scleral anatomical geometry; (5) the ocular device employs one or more lobes to maximize the mass and volume; (6) the ocular device employs two lobes with greater mass and thickness than the central connecting portion (dumbbell shape); (7) the ocular device has a volume from about 70 μm to about 400 μm; (8) the ocular device has a length from about 8 mm to about 35 mm; (9) the ocular device has a height from about 1.0 mm to about 14 mm; (10) the ocular device has a thickness from about 0.10 mm to about 5.0 mm; (11) the ocular device has a defined edge apex contour; (12) the ocular device has a defined edge lift; (13) the ocular device has a defined front curve(s); (14) the ocular device has front curves that are tonic; (15) the ocular device has front curves that are aspheric; and (16) the ocular device has a lenticular that is utilized on the front curve geometry.

The present invention can be made in considerably larger dimensions than is claimed by prior art, and yet still remain stable and comfortable. The consequent volume, shape features and intended use of the device design renders its insertion, in situ evaluation and removal intuitive to the ophthalmologist, optometrist, other contact lens practitioner, nurse, or ophthalmic technician. The present invention describes a device that does not need forceps or other instruments or surgical procedures for insertion or removal. Patients could be taught to insert and remove such a device, in the manner that they are taught to insert and remove contact lenses. This does not preclude the device from being placed underneath the conjunctiva or Tenon's capsule, for example, for drug delivery to the posterior segment of the eye, in which case surgical instruments would be involved in the procedure of device implantation.

In one preferred embodiment, the devices are made of non-erodable or erodable materials. Examples of non-erodable materials are, but are not limited to, polyacrylates and methacrylates, polyvinyl ethers, polyolefins, polyamides, polyvinyl chloride, fluoropolymers, polyurethanes, polyvinyl esters, polysiloxanes and polystyrenes. Examples of erodable materials are cellulose derivatives such as methylcellulose, sodium carboxymethyl Cellulose, hydroxyethyl cellulose, hydroxypropyl cellulose and hydroxypropylmethyl cellulose; acrylates such as polyacrylic acid salts, ethylacrylates and polyacrylamides; natural products such as gelatin, collagen, alginates, pectins, tragacanth, karaya, chrondrus, agar and acacia; starch derivatives such as starch acetate, hydroxyethyl starch ethers and hydroxypropyl starch as well as synthetic derivatives such as polyvinylalcohol, poly vinylpyrrolidone, poly vinyl methyl ether, poly ethyleneoxide, neutralized Carbopol®, xanthan gum, polyester, poly ortho ester, poly anhydride, poly phosphazine, poly phosphate ester, poly caprolactone, poly hydroxybutyric acid, poly glycolic acid, poly lactic acid and combinations thereof.

In another embodiment of the present invention, there is provided a method of delivering a drug to the eye of an individual in need of such medication, comprising the steps of placing the drug into the drug delivery device and then contacting the individual with the drug-containing drug delivery device by placing the device on the inferior or superior sclera of the eye. A representative ocular disease is glaucoma; those skilled in the art will recognize other diseases, infections or inflammations of the eye that could be treated in this manner using this invention. The drug delivery devices of this invention may contain any of a variety of useful drugs, for glaucoma, allergy, infection, inflammation, uveitis, trauma, post-surgical prophylaxis, pain, dry eye or degenerative conditions. Other agents, such as lubricants, humectants, viscosifiers, demulcants or vitamins, may also be delivered with this invention.

In yet another embodiment of the present invention, there is provided a method of delivering a drug systemically to an individual in need of such medication, that includes the steps of: placing a drug with poor ocular absorption kinetics into the drug delivery device and then contacting the individual with the drug-containing drug delivery device by placing the device on the inferior or superior sclera of the eye so that the drug that is released travels with the tear drainage pathway into the naso-lacrimal duct and is absorbed systemically via the nasal mucosa and drainage pathway. A representative systemic disease is diabetes, and a representative drug is insulin; those skilled in the art will recognize other systemic diseases, infections or inflammations that could be treated in this manner using the present ocular devices.

In another embodiment of the present invention, there is provided a method of delivering a drug to the eye of an individual in need of such medication, comprising the steps of placing the drug into the drug delivery device and then contacting the individual with the drug-containing drug delivery device by placing the device on the inferior or superior sclera of the eye posterior to the superior or inferior rectus muscle insertions, below the conjunctiva, intermuscular membrane or Tenon's capsule, or even into the episcleral space. In this surgical implantation, the device would still provide a large volume in a shape corresponding to the anatomical potential space of insertion. Movement with eye movement would be limited and less necessary than for embodiments worn on the external eye. The posterior eye would be more accessible for drug penetration from this embodiment as placed. Representative ocular diseases are macular degeneration, posterior uveitis, endophthalmitis, diabetic retinopathy, glaucomatous neuropathy; those skilled in the art will recognize other diseases, infections or inflammations of the posterior eye that could be treated in this manner using this invention. The drug delivery devices of this invention may contain any of a variety of useful drugs, for glaucoma, retinopathy, infection, inflammation, uveitis, trauma, post-surgical prophylaxis or degenerative conditions. In another embodiment of the present invention, there is provided a method of delivering a drug systemically to an individual in need of such medication, comprising the steps of placing the drug into the drug delivery device and then contacting the individual with the drug-containing drug delivery device by placing the device on the inferior or superior sclera of the eye. A representative systemic disease is diabetes; those skilled in the art will recognize other diseases, infections or inflammations of the body that could be treated in this manner using this invention. In another embodiment of the present invention, there is provided a method of delivering a drug systemically to an individual in need of such medication, comprising the steps of placing the drug into the drug delivery device along with an electrode and appropriate membrane, and then contacting the individual with the drug-containing drug delivery device by placing the device on the inferior or superior sclera of the eye. A discrete amount and time of charge is then applied resulting in immediate delivery of a dose of drug via the process of iontophoresis. This process is then repeated with the same device left in place until depleted of drug or after placement of a new device for each dosing.

One important embodiment of this invention concerns compounds and drugs that exhibit poor solubility in aqueous systems. For that reason poor solubility of drugs is a challenge in drug formulation and a common task in pharmaceutical companies is overcoming these solubility problems. It is well known that control of drug particle size is a means of controlling the rate of dissolution. Particle size will not affect the equilibrium solubility of a drug but in systems where the dissolving drug is carried away, a condition known as the infinite sink, then the rate of drug dissolution becomes important. This is especially true for poorly water-soluble drugs. Therefore, the bioavailability of low solubility drugs is often intrinsically related to drug particle size. By reducing particle size, the increased surface area may improve the dissolution properties of the drug to allow a wider range of formulation approaches and delivery technologies. Conventional methods of particle size reduction are comminution and spray drying and rely upon mechanical stress to disaggregate the active compound. The critical parameters of comminution are well known to the industry, thus permitting an efficient, reproducible and economic means of particle size reduction.

This embodiment of the invention describes the use of hydrogel systems for the delivery of drug or agent that is poorly soluble in water to the eye to treat a disease or condition. Furthermore, the delivery system of this embodiment of the invention delivers drug in a sustained manner over long periods of time. This embodiment of the invention utilizes poorly water soluble drugs or agents in the form of micronized or nanosized particles exhibiting very large surface areas. The poorly water-soluble drugs utilized in this embodiment of the invention have solubility's ranging from one mg per ml down to nanograms per ml of water. Those skilled in the pharmaceutical art will recognize the methods available to reduce the particle size of solids such as drugs. Particle size reduction can be carried out “dry” or ‘wet’. The recovery of particles in the dry state is most common but in some cases particle size reduction is carried out in the presence of water. In this case the drug is processed through a mill that uses water jets to reduce particle size. Often times a surfactant or dispersion aid is included to prevent particle agglomeration and to obtain a semi-stable suspension of the drug in the water.

This embodiment of the invention utilizes a hydrogel material as the body of the delivery device. The hydrogel may be the conventional polyhydroxyethyl methacrylate type or the newer silicone hydrogels. Both of these types of hydrogels are the basis for soft contact lenses. The equilibrium water content for the hydrogels useful in this embodiment of the invention can range from about 5% to about 70%. Most useful are hydrogels that have water content of between 5% and 55%. For very poorly soluble drugs the higher water content hydrogels are preferred while for more soluble drugs the lower water content hydrogels are preferred. The polymeric device is prepared from monomers plus a free radical initiator into which the micronized or nanosized drug has been dispersed. The formulation, in the form of a suspension, is placed in a device mold, and the polymerization is carried out thermally. Optionally, the micronized or nanosized drug is dispersed, often times in the presence of a surfactant or protective colloid, in water and then added to the monomers containing a free radical initiator. In either case after polymerization the device can be placed in water or saline to achieve its equilibrium water content.

The drug delivery systems described above are classified as matrix systems containing a dispersed drug. In these systems the drug must first dissolve into the matrix then diffuse through the matrix to be released into the ocular environment. Given a poorly soluble drug this process would normally be extremely slow with little drug released. This embodiment of the invention describes a hydrogel matrix with variable water contents. Hydrogels contain interconnected water channels throughout the matrix that act as “rivers” for the transport of solublized drug. The drug particle is in contact with the water phase in the hydrogel and thus the drug dissolves directly into the water phase for easy transport and release from the device. The large surface area of the particles provides for an increased rate of drug solubility into the water channels. The release rate of drug from the device matrix can be controlled by the amount of water in the hydrogel matrix. Low water containing hydrogels have less and smaller water channels thus impeding the transport of the drug and lowering the overall release rate.

The hydrogels described above are fashioned into the devices described in this embodiment of the invention. Additionally, these devices can be implanted in the eye for treatment of back-of-the-eye diseases. For example the device can be placed under the conjunctiva.

In another important embodiment the present invention provides topical ocular drug delivery devices, systems and methods for sustained delivery of a prostaglandin analogue to the ocular tissues of the patient for the treatment of glaucoma. In particular, this embodiment of the invention describes a topical delivery device and method for the prostaglandin analogue drugs such as latanoprost, travaprost and bimatoprost.

The construction of a drug delivery device of this embodiment of the invention requires that the stability limitations of prostaglandin analogues be addressed. For this reason the delivery device must be formed in two distinct operations.

The first operation is the formation of the device body. For example, the device may be injection molded from a thermoplastic material. One material for this purpose would be ethylene vinyl acetate although many other materials would be acceptable. Another method for generating the device body is cast molding, a standard process for the production of soft contact lenses. In this process a liquid monomer mix is cast into a two-piece plastic mold. The mold or “casting cups” are usually injection molded polypropylene (see Examples 5 and 6). The polymerization process can be carried out by the application of heat and/or ultraviolet radiation. Once cured the polymerized device is removed from the mold.

In the case of a device for the sustained delivery of prostaglandin analogues certain modifications to the device body are necessary. These modifications are necessary due to two factors: firstly, only small amounts, nanograms per day, of prostaglandins are required for an effective glaucoma treatment; and secondly prostaglandins are relatively costly. Therefore only a small amount of a prostaglandin analogue is required in each device of this invention. To conserve costs, yet provide the proper drug release rate, the prostaglandin analogue should be localized in the device. To accomplish this localization of drug the basic device body is molded or cast with small cavities or “holes” in either the “top”, (distal surface of the device), or the “bottom”, (proximal surface of the device), or both surfaces. These cavities are preferably circular “holes” and can range in diameter from a fraction of a millimeter to millimeters. The depth of these cavities or “holes’ can also range in depth from a fraction of a millimeter to millimeters. The cavities or “holes” in one device can vary both in number and position on the surface. These cavities or “holes” will serve as a type of reservoir for the prostaglandin analogue drug.

The second operation in the construction of the drug delivery devices of this embodiment of the invention is the introduction of the prostaglandin analogue into the cavitity of the device. The prostaglandins are generally oily or waxy substances and therefore not suited for direct placement into the cavities on the device. Rather the prostaglandin should be placed in a carrier matrix that is elastomeric in nature and non-biodegradable. Because of the stability issues with the prostaglandin analogues this procedure must be carried out at near room temperature with materials that will not cause degradation of the drug. One such matrix material is an RTV silicone rubber, formed from a silicone liquid that is cured at room temperature. It should be noted that many other materials could also serve as the matrix for the prostaglandin analogues. Silicones are particularly useful since they allow for the permeability of many drugs and have been used commercially as the body for drug delivery devices. In practice the prostaglandin analogue would be mixed with a silicone formulation resulting in a fine dispersion of the prostaglandin analogue. While a small amount of the prostaglandin analogue may be soluble in the silicone formulation the bulk of the drug would be dispersed as fine “droplets”. This type of system is referred to as a dispersed matrix system. Once the silicone/drug formulation is mixed it would be placed in the cavities on the surface of the device body. After the silicone cures into a rubber the device is complete and ready for use. It should be noted that the number of cavities, the open area of the cavities and the concentration of the drug in the matrix govern the release rate of the prostaglandin analogue from the devices of this invention. The depth of the cavities governs the duration of release.

The above described device contains, for example, a silicone/drug core that allows the prostaglandin analogue to diffuse out from the cavities into the tear fluid, as desired, but also allows the drug to diffuse from the core sides and bottom into the device body. In some cases this non-productive route may lead to negligible drug loss and therefore is of no consequence. However, in cases where this loss is of consequence the wasteful loss of drug results in a negative impact on device cost due to the high price of the prostaglandin analogues.

The devices of this invention can be made more efficient by imposing uni-directional diffusion of the prostaglandin analogue directly to the tear fluid. This avoids prostaglandin loss from the sides of the drug core. A method of providing uni-directional diffusion of a prostaglandin analogue from the drug/matrix core is to form the drug/matrix core inside a sheath. This sheath is preferably a plastic.

Furthermore the plastic tube should be formed from a material that is substantially impermeable to the prostaglandin analogue. Examples of such materials are the polyolefins such as polyethylene and polypropylene.

A section of prescribed length of the encapsulated drug/matrix will then serve as the delivery portion of the devices of this invention. This section would be fitted into a cavities or cavities that were created on the device body as described above. The release of the prostaglandin analogue would then be from the area of the tube exposed to the ocular environment. The other end of the tube would be in contact with the device body and provide a substantial barrier to diffusion of the prostaglandin analogue into the device itself. A preferred construction would place the prostaglandin analogue on the “bottom” (proximal surface of the device) so that the drug is released towards the sclera to provide a more direct route to the eye itself.

The encapsulated drug/matrix core can be designed to accelerate or retard the release of the prostaglandin analogue without changing the diameter of the tube itself. For example, by increasing the surface area of the drug/matrix core the release rate can be increased. The drug/matrix core can have a hollow center.

Another example would be the retardation of the release of the prostaglandin analogue without changing the diameter of the tube itself. For example, by decreasing the surface area of the exposed drug/matrix core the release rate can be slowed. The drug/matrix core with a restricted opening would accomplish this.

In summary, the prostaglandin analogue releasing devices of this embodiment of the invention are prepared in two steps. The first step involves producing a device body with “holes” or cavities present on either or both surfaces. There may be only one cavities present or more than one cavities present. In a second step the drug/matrix is prepared by dispersing the prostaglandin analogue in a polymer matrix as a dispersed phase. This drug/matrix is placed directly into the cavities located on the device body. Alternatively, the drug/matrix is placed in a tube and a section of that tube is placed into the cavities located on the device body.

One important aspect of this embodiment of the invention relates to the construction of a device that delivers another glaucoma drug in conjunction with the delivery of a prostaglandin analogue. The device would be termed a “combination” delivery system. In the first step the body of the device would be prepared with a glaucoma drug dissolved or dispersed in the body itself. Then in the second operation the prostaglandin analogue would be introduced to the device as previously described. An example of such a combination device would be a device that contains timolol dissolved in the device body with cavities that contain latanoprost. The device would then release, in tandem, both timolol and latanoprost at sustained rates over long periods of time.

It is an object of this invention to describe various ocular applications for a controlled topical drug or agent delivery to the eye for enhanced treatment of a disease or condition. These applications are, but not limited, to the following:

Glaucoma

Allergy

Infection
Bacterial
Fungal
Virus

Inflammation

Post-surgical prophylaxis

Pain

Trauma

Dry eye

AMD

Diabetic macular edema

Uveitis

Retinitis

It is also an object of this invention to describe the various drugs and agents delivered to the eye, in a controlled manner, for enhanced treatment of a disease or condition. It should be noted that the term “drugs and agents”, for the purpose of this invention, will also be expressed collectively as “therapeutic agents”.

There are a wide variety of drugs and agents available to treat the various aforementioned ocular diseases and conditions. It should be noted that any suitable ocular drug or agent, for a particular application, can be administered in a controlled manner in accordance with the practice of this invention. It should also be noted that combinations of drugs and/or agents can delivered to the eye in a controlled manner in the practice of this invention.

It is another object of this invention to describe the various mechanisms for the controlled topical drug or agent delivery to the eye for enhanced treatment of a disease or condition. These mechanisms include:

Physical or physiochemical systems

Chemical or biochemical

A combination of the above two systems

The physical or physiochemical systems include reservoir systems, matrix or monolithic systems, swelling-controlled systems or hydrogels, and osmotic systems or osmotic pumps or a combination of these processes.

The chemical or biochemical systems are biodegradable polymeric compositions that can be degraded at the site of installation. The degradation of the polymer may be through hydrolysis, enzyme attack or microorganism breakdown, or a combination of these processes.

It is yet another object of this invention to describe the various polymeric materials that are useful as carriers for controlled topical drug or agent delivery to the eye for enhanced treatment of a disease or condition. These materials are polymeric in nature and can be chosen from, but not limited, to the following non-erodible and erodible materials or combinations of the two classes.

Examples of non-erodable materials are, but are not limited to, polyacrylates and methacrylates, polyvinyl ethers, ethylene vinyl acetate and alcohol, cellulosics, polybutylenes, polyolefins, polyamides, polyvinyl chloride, fluoropolymers, polyurethanes, polyesters, polyvinyl esters, polysiloxanes, thermoplastic elastomers; and polystyrenes and combinations thereof.

Examples of erodible materials are cellulose derivatives such as methyl cellulose, sodium carboxymethyl cellulose, hydroxyethyl cellulose, hydroxypropyl cellulose and hydroxypropylmethyl cellulose, polyacrylates such as polyacrylic acid salts, methylacrylates and polyacrylamides; natural products such as gelatin, collagen, alginates, pectins, tragacanth, karaya, chrondrus, agar and acacia; starch derivatives such as starch acetate, hydroxyethyl starch ethers and hydroxypropyl starch as well as synthetic derivatives such as polyvinylalcohol, poly vinylpyrrolidone, polyvinyl methyl ether, polyethyleneoxide, polypropyleneoxide, neutralized Carbopol®, xanthan gum, polyesters, poly(ortho esters), poly anhydride, poly phosphazine, poly phosphate ester, polycaprolactone, polyhydroxybutyric acid, polyglycolic acid, polylactic acid and combinations thereof.

The ocular delivery devices of this invention can be fabricated from polymer based materials. The drug or medicinal agent can either be in a dissolved or dispersed state within the polymeric matrix. The devices of this invention can then be fabricated from these materials by any of the standard conversion techniques such as injection molding, compression molding or transfer molding. In another embodiment, the drug or medicinal agent can be compounded into a reactive system. That system may be a monomer or macromer where the drug or medicinal agent is in the dissolved or dispersed state. Polymerizing the system through UV, visible light, heat or a combination of these means then forms the device. Examples would include the use of liquid acrylic monomers or a reactive silicone pre-polymer.

A preferred manufacturing process for producing the drug delivery devices of this invention is cast molding. In this process a drug is dissolved or dispersed in a monomer mixture and placed in a plastic casting mold bearing the geometry of the ocular device. Thermal exposure, UV exposure or a combination of both polymerizes the monomer. The device is then removed from the mold. Post processing may be required, for example edge finishing. In the case of an ocular device polypropylene casting molds are preferred. Most preferred is a polypropylene resin with a melt flow index above 20. One polypropylene resin is Exxon PP1105E, which has a melt flow index of 34 g/10 min. With melt flows above 20 gm/10 min intricately shaped casting molds can be injection molded with excellent replication of part dimensions.

Post processing is oftentimes required to remove flash and/or to contour the parting line. For an ocular device, such as contact lenses and the devices of this invention, the edge profile is critical in providing device comfort and fit. The edges of the ocular devices of this invention can be shaped and contoured utilizing standard polishing techniques currently available for rigid gas permeable contact lenses. More preferred is the use of cryogenic deburring to form a smooth, well-contoured edge.

An aspect of the present invention may also be described as a therapeutic package for dispensing to, or for use in dispensing to, a mammal being treated for a medical condition, disorder or disease. In the case of a device utilized to treat an ocular condition or disease the therapeutic package comprises:
(1) A medical device containing a prescribed amount of a medicinal agent packaged in a container, which is constructed from either glass or plastic. The device may be either in a sterile or a non-sterile state within the package. The dosage form contains sufficient medicinal agent that is effective to lessen, stabilize or eradicate medical conditions, disorders or diseases when administered over a defined period of time.
(2) A finished pharmaceutical container or package therefore, said container containing
   (a) a medical device containing a medicinal agent
   (b) labeling directing the use of said package in the treatment of said mammal

The compositions of this invention in the form of a medical device containing medicinal agent, for the continuous, sustained release of said medicinal agent can be packaged in an appropriate container. The physician or the patient would utilize the packaged product in accordance with the prescribed regimen. Typically, in the case of an ocular device the physician would insert the device under the upper or the lower eyelid. In other cases, the patient would insert the device under the upper or the lower eyelid. The ocular device would be maintained, in place, for the prescribed period of time. The product container and associated packaging will bear identification, information and instructions in accordance with local, federal and foreign governmental regulations. The inclusion of a “package insert” is also generally required. The “package insert” will provide information pertaining to contents, action, indications, contraindications, warning, how supplied, safety information and precautions, as well as directions for use.

### Example 1

The aspects of the device of Example One are shown in FIGS. 6-8. The overall shape of this invention is greater horizontally than vertically, and can appear as an oval in as shown in the front elevation view of FIG. 6. It is preferred that the shape be symmetrical about the vertical meridian, such that the lateral halves are mirror images. This aspect allows for the same device design to be used in the right and left eyes (in the same orientation), and on the superior or inferior sclera of an eye. The base curve 114 radius is chosen to fit the sclera 50. The center thickness is greatest in the horizontal centerline, with tapering to a defined minimal, mostly uniform edge thickness around the entire edge perimeter of the ellipse where the anterior surface 207 and posterior surface, 209 meet. This entails a significantly tonic shape on a fairly spherical base curve with a uniform edge radius. Size can range from about 10 mm to about 25 mm in width by about 5 mm to about 12 mm in ht by about 1.0 mm to about 3.0 mm center thickness. The base curve radius 114 is from about 10 mm to about 20 mm. The volume of the device ranges from about 70 μm to about 400 μm. A device in accordance with FIGS. 6-7 was constructed from a silicone elastomer. The dimensions were 16 mm in width, 7.0 mm in height and 2.3 mm in center thickness, which tapered down from the center point. The tone front surface radii were 4.0 mm vertical meridian by 9.0 mm horizontal meridian. The base curve radius was 12.4 mm. The device volume was 150 μm.

### Example 2

The aspects of the device of Example two are shown in FIGS. 6-8. The general geometric parameters were discussed in Example One. A prototype device was constructed from silicone elastomer. The overall width was 21.0 mm, the height was 7.8 mm and the center thickness was 1.5 mm. The toric front surface radii were 5.0 mm vertical meridian and 12.0 mm horizontal meridian. The base curve radius was 12.4 mm. The overall device volume was 150 μm. This device was placed on the superior sclera of a subject's eye. The device was stable in the eye with slight rotation observed. The comfort of the device was reported to be good.

### Example 3

The aspects of the device of Example Three are shown in FIGS. 6-8. The general geometric parameters were discussed in Example One. A prototype device was constructed from silicone elastomer. The overall width was 24.5 mm, the height was 10.0 mm, and the center thickness was 2.3 mm. The toric front surface radii were 6.0 mm vertical meridian by 12.5 mm horizontal meridian. The overall device volume was 385

The device was placed on the superior sclera of a subject's eye. The device tended to move slightly to a nasal position. The comfort was rated at “slight awareness”.

### Example 4

The aspects of the device of Example Four are shown in FIGS. 9-12. The overall shape is a horizontal “dumbbell” symmetrical about both the central vertical axis and the central horizontal axis. A prototype device that included the lenticular feature on the anterior geometry of the lobes was constructed from silicone elastomer. The distance between the anterior and posterior surfaces, center thickness, (midway between the lobes) was 0.75 mm. The distance between the two surfaces at the center of each lobe was 1.5 mm. The anterior curvature at the center of the lobe was 4.3 positive radius, transitioning to 2.0 mm negative lenticular radius and then transitioning to a 0.25 positive edge radius. Overall width was 20.5 mm. Vertical height was 8.45 mm at its maximum at each lobe, and 6.5 mm at the center of the device. The back curve radius was approximately 12.4 mm. Volume was 130 μm. The lobes could be detected (cosmetically visible) as slight elevations of the eyelid. The device with the lenticular demonstrated clinically acceptable position, stability and retention, both in the superior and inferior positions. Comfort was quite good, with the exception of some sensation of the edge.

### Example 5

The aspects of the device of Example Five are shown in FIGS. 13-15. A prototype device was made that was overall higher and wider than Example 4. This device was 21 mm wide and 7.25 mm height in the center of the device. This dumbbell version was 9.5 mm in the dumbbell lobe height as viewed from the front. A uniform spherical 12.4 mm back curvature was used, as the material used was quite flexible. The indentation distal to the cornea yielded a 0.26 mm maximum differential in height of the device due to this curvature. Device was 2.77 mm from the horizontal meridian running through the center of the peripheral lobes to the edge of the device proximal to the cornea. The same measurement from the horizontal meridian (running through the center of the peripheral lobes) to the edge was 4.47 mm on the side distal to the cornea. We increased the front negative lenticular curvature to 2.1 mm. The actual true radius was therefore 4.0 mm. We smoothed over the transition curves to make the “bumps” of the lobes less visible under the upper lid during wear. The width is slightly greater as well. The anterior edge radius was decreased, bringing it more into the realm of a contact lens radius but the edge lift was the same. The tighter radius is an attempt to lessen the edge sensation from the upper lid, to increase comfort. Volume was 136 μm.

On eye, this device was the most comfortable yet in the superior position. No “bumps” were visible under the superior lid. It felt very stable in its interaction the lid. Removal was still relatively easy to accomplish by massaging the device downward via external manual manipulation of the eyelid and then removing the device manually, as is done with a contact lens, once it became visible in the palpebral aperture.

### Example 6

The aspects of the device of Example Six are shown in FIGS. 13-15. A prototype device was cast-molded from an acrylic monomer, with increased edge lift compared to Example 5 due to the addition of a secondary peripheral curve radius. This device was 21 mm wide and 7.25 mm in height in the center of the device. This embodiment was 9.45 mm in the height of the lobe sections as viewed from the front. The horizontal front curve is a spline that smoothly blends the center and lobe regions that have defined vertical front curve radii and edge lift radii and widths. The front curvature radius in the center axis 15-15 was 7.26 mm centrally, and 5.09 mm at the lobes. The indentation proximal to the cornea was cut at a lenticular radius of 0.75 mm and yielded a 1.95 mm maximum differential in height of the device due to this curvature. The device was 2.77 mm from the axis 14-14 running through the center of the peripheral lobes to the edge of the device proximal to the cornea. The indentation distal to the cornea was cut at a lenticular radius of 1.50 mm and yielded a 0.26 mm maximum differential in height of the device due to this curvature. The device was 4.47 mm from the axis 14-14 running through the center of the peripheral lobes to the edge of the device distal to the cornea. The lenticular reverse curve of the lobe was 2.1 mm. The width of the lenticular curve was 1.13 mm proximal to the cornea and 1.23 distal to the cornea. The edge apex radius was 0.56 mm with an edge thickness of 0.43 mm. A toric-12.4 mm vertical meridian (axis 15-15), 12.5 mm horizontal meridian (axis 14-14)—back curvature was used since the material was quite flexible. The edge lift base curve radius was 16.4 mm, with a width of 1.0 mm, in the vertical meridian centrally (15-15), and 16.4 mm, with a width of 1.2 mm, along the entire periphery at the lobes. The volume was 124 μm.

The ocular device of this Example 6 was cast-molded from an acrylic monomer formulation as follows. The design of the device was machined into metal molds. Casting mold halves were injection molded from Exxon polypropylene PP1105E. Under an inert atmosphere the lower casting mold half was filled with an acrylic monomer formulation containing a UV initiator. The upper casting mold half was fitted into the lower casting mold half to form the device shape. The closed casting mold assembly was placed in a UV curing chamber and exposed to UV at wavelength 365 nm for thirty minutes. The polymerized ocular device was then removed. A peripheral curve system was molded into the posterior periphery of the device. Their width and their incremental increases in radius values define these peripheral curves over the central base curves. In one embodiment, these values for each curve can be uniform around the peripheral posterior surface of the device. Our most preferred peripheral curve system comprises curves of different widths in the central and lateral lobe parts of the device. The peripheral curve system provides the edge lift. This approach is utilized in the contact lens art to enhance comfort, movement and tear film exchange. When placed on a subject, the device of this Example 6 performed as well as that of Example 6 in all aspects, with the additional results of having increased comfort with little or no sensation of the device in the eye. Lag with eye movement, and movement and repositioning with blink, were excellent. Utilizing a fluorescent dye, a peripheral band of dye under the device, corresponding to the peripheral curve system and its associated edge lift, could be observed in a manner consistent with standard clinical evaluation of such an observation of rigid contact lenses. The width, evenness, and intensity of this band of fluorescent dye, relative to the fluorescent intensity under the rest of the device, was judged to be clinically excellent using criteria practiced by one skilled in rigid contact lens clinical practice.

### Example 7

A topical ocular device of this invention for the treatment of dry eye signs and/or symptoms by releasing the therapeutic agent(s) continuously over time is described here. The ocular device is classified as a physical or physicochemical system. Such systems include reservoir systems, matrix or monolithic systems, swelling-controlled systems.

The polymer matrix can be tailored to the particular therapeutic agent chosen for delivery to the eye. For example, if Cyclosporin is the drug chosen for continuous delivery to the eye the polymer matrix may formulated to provide little or low water content (about 5% or less). In this manner the Cyclosporin can be delivered to the eye continuously for days, weeks or months.

If a water soluble lubricant, such as glycerin, is chosen for continuous delivery to the eye then the polymer matrix chosen may be a hydrogel. If the matrix formulation is composed of about 62% hydroxyethyl methacrylate and about 38% glycerin the resultant polymer will be a clear, rubbery material in the form of the device design described above in Examples 5 and 6. When placed in the eye the glycerin will slowly diffuse out of the device to provide continuous lubrication of the ocular surface. At the same time water will diffuse into the device replacing the glycerin resulting in little, or no, dimensional changes in the device geometry.

### Example 8

A topical ocular device for the treatment of dry eye signs and/or symptoms by releasing the therapeutic agent(s) continuously over time is described herein. Said ocular device being classified as biodegradable polymer systems—this category includes biodegradable polymeric systems and bioadhesive systems.

Said ocular devices are constructed of polymers that can be degraded at the place applied. For example, the polymer degradation may occur in the eye. The degradation of polymers may be accomplished through simple solvation, hydrolysis, enzyme attack, or microorganism breakdown. Some examples of erodible materials would include, but not limited to, cellulose derivatives such as methyl cellulose, sodium carboxymethyl cellulose, hydroxyethyl cellulose, hydroxypropyl cellulose and hydroxypropylmethyl cellulose, polyacrylates such as polyacrylic acid salts, methylacrylates and polyacrylamides; natural products such as gelatin, collagen, alginates, pectins, tragacanth, karaya, chrondrus, agar and acacia; starch derivatives such as starch acetate, hydroxyethyl starch ethers and hydroxypropyl starch as well as synthetic derivatives such as polyvinylalcohol, poly vinylpyrrolidone, polyvinyl methyl ether, polyethyleneoxide, polypropyleneoxide, neutralized Carbopol®, xanthan gum, polyesters, poly(ortho esters), poly anhydride, poly phosphazine, poly phosphate ester, polycaprolactone, polyhydroxybutyric acid, polyglycolic acid, polylactic acid and combinations thereof.

In the simplest case the entire device may be constructed of a water soluble/erodible polymer. When placed in the eye the device begins to “dissolve” releasing polymer into the tear film. The polymer acts as a lubricant for the ocular surface. Useful polymers for this purpose would include, but not limited to, hydroxypropyl cellulose and hydroxypropylmethyl cellulose. By the choice of polymer(s) utilized to construct the device solubility may be controlled to provide a device that releases lubricating polymer for days, weeks or longer.

A variation of the above method would include a drug, such as Cyclosporin, within the soluble/erodible polymer matrix. In this manner both lubrication and a therapeutic treatment can be performed simultaneously. The patient then benefits from two methods of therapy.

In another mode of this invention the release of therapeutic agent(s) can be maintained for long periods of time. In this case a bio-erodible polymer is employed, one that erodes over weeks and months. For example, incorporation of a drug such as Cyclosporin into a bio-erodible matrix would provide slow drug release over a prolonged period of time as treatment for dry eye. Useful polymers for this purpose would include, but not limited to, polyesters, poly (ortho esters), poly anhydride, poly phosphazine, poly phosphate ester, polycaprolactone, polyhydroxybutyric acid, polyglycolic acid, polylactic acid and combinations thereof.

### Example 9

A topical ocular device for the treatment of dry eye signs and/or symptoms by releasing the therapeutic agent(s) continuously over time is described herein. Said ocular device being classified as a composite polymeric material comprising a:
1. A non-erodible polymer(s)
2. An erodible polymer(s)

The two polymer phases both have exposure at the surface of the device, that is, one polymer is not internal to the other.

Said ocular device is a composite composed of: (1) A non-erodible polymeric material(s), preferably a polymer material(s) with a glass transition temperature below about 35° C. Examples of non-erodable materials are, but are not limited to, polyacrylates and methacrylates, polyvinyl ethers, ethylene vinyl acetate and alcohol, cellulosics, polybutylenes, polyolefins, polyamides, polyvinyl chloride, fluoropolymers, polyurethanes, polyesters, polyvinyl esters, polysiloxanes, thermoplastic elastomers, and polystyrenes and combinations thereof and (2) . . . . An erodible polymer(s) that can be degraded at the place applied. For example, the polymer degradation may occur in the eye. The degradation of polymers may be accomplished through simple solvation, hydrolysis, enzyme attack, or microorganism breakdown. Some examples of erodible materials would include, but not limited to, cellulose derivatives such as methyl cellulose, sodium carboxymethyl cellulose, hydroxyethyl cellulose, hydroxypropyl cellulose and hydroxypropylmethyl cellulose, polyacrylates such as polyacrylic acid salts, methylacrylates and polyacrylamides; natural products such as gelatin, collagen, alginates, pectins, tragacanth, karaya, chrondrus, agar and acacia; starch derivatives such as starch acetate, hydroxyethyl starch ethers and hydroxypropyl starch as well as synthetic derivatives such as polyvinylalcohol, poly vinylpyrrolidone, polyvinyl methyl ether, polyethyleneoxide, polypropyleneoxide, neutralized Carbopol®, xanthan gum, polyesters, poly(ortho esters), poly anhydride, poly phosphazine, poly phosphate ester, polycaprolactone, polyhydroxybutyric acid, polyglycolic acid, polylactic acid and combinations thereof.

One useful device in the design described above in Examples 5 and 6 would be a composite material of a non-erodible base, that is the portion of the device that contacts the sclera, combined with an erodible material on the top surface of the device, that is the portion of the device that contacts the eye lid. In the simplest case the erodible material would be a water soluble/erodible polymer such as, but not limited to, hydroxypropyl cellulose and hydroxypropylmethyl cellulose. The non-erodible base would provide stability and comfort while the device is in the eye. While the erodible material provides continuous lubrication to the eye by the choice of erodible polymer(s) utilized to construct the device polymer solubility may be controlled to provide a device that releases lubricating polymer for days, weeks or longer.

In keeping with the above described composite device a variation of the above method would include a drug, such as Cyclosporin, within the water soluble/erodible polymer matrix. In this manner both lubrication and a therapeutic treatment can be performed simultaneously. The patient then benefits from two methods of therapy.

In another mode of the above described composite device the release of therapeutic agent(s) can be maintained for long periods of time. In this case a bio-erodible polymer is employed, one that erodes over weeks and months. Incorporation of a drug such as Cyclosporin into a bio-erodible matrix would provide slow drug release over a prolonged period of time. Useful polymers for this purpose would include, but not limited to, polyesters, poly (ortho esters), poly anhydride, poly phosphazine, poly phosphate ester, polycaprolactone, polyhydroxybutyric acid, polyglycolic acid, polylactic acid and combinations thereof.

### Example 10

The following experiment was designed to create a device of this invention that releases a lubricant continuously over at least one day. The approach is to polymerize the lubricant into a hydrogel matrix. The resultant device will then release the lubricant into the ocular environment. The lubricant will be replaced with water from the tears. The following example illustrates this type of device.

A base formulation was prepared as follows:

| | | |
|---|---|---|
| Hydroxyethyl Methacrylate | 100 | ml |
| Polyethyleneglycol Dimethacrylate | 1 | ml |
| Photoinitiator SR-1129 | 0.5 | gms |

The above base formulation is mixed with the glycerin in the following proportions:

| | |
|---|---|
| Base Form | 60 parts |
| Glycerin | 40 parts |

The final formulation is pipetted into the base half of the polypropylene molds as described in Examples 5 and 6. The second mold half, the cover, is fitted into the mold base to seal off the formulation and form the desired device geometry. The filled molds are placed in a UV curing chamber, Model CL-1000L available from UV Process Supply, Inc, such as Model CL-1000L available from UV Process Supply, Inc. This chamber operates at a UV wavelength of 365 nm. To accomplish polymerization, the UV exposure energy was set at 120,000 micro joules per cm² and the exposure time was 30 minutes. The resulting device was clear and exhibited a degree of flexibility. When placed in saline buffer the glycerin containing device maintained its shape and volume for several days while the glycerin was releasing from the device matrix.

### Example 11

The following formulation produces a polymer matrix that contains timolol free base in the dissolved state. When cast/molded into a device of this invention the resulting ocular drug delivery system releases timolol in a controlled manner over several months. This system is well suited to the treatment of the ocular disease glaucoma

| INGREDIENT | AMOUNT (gms) |
|---|---|
| Di(ethyleneglycol)ethylether | 57.91 |
| methacrylate | |
| 2,2,2-Trifluoroethyl | 31.56 |
| methacrylate | |
| Polyethyleneglycol(330) | 4.81 |
| dimethacrylate | |
| Methacrylic acid | 0.91 |
| Timolol Free Base | 4.45 |
| Esacure KIP 100F | 0.36 |

The monomers were purified to remove inhibitors prior to formulation preparation. The above formulation, containing a UV initiator, is placed in a vial then purged with nitrogen to remove oxygen. The vial was quickly stoppered to exclude reintroduction of oxygen. The stoppered vial of formulation is placed in a glove box along with the two piece polypropylene mold halves described in Examples 5 and 6. The glove box is then purged with nitrogen to remove oxygen. Once this has been accomplished the formulation is opened and a prescribed amount of formulation is pipetted into the base half of the polypropylene mold. The second mold half, the cover, is fitted into the mold base to seal off the formulation and form the desired device geometry. The filled molds are placed in a UV curing chamber, such as Model CL-1000L available from UV Process Supply, Inc. This chamber operates at a UV wavelength of 365 nm. To accomplish polymerization, the UV exposure energy was set at 120,000 micro joules per cm² and the exposure time was 30 minutes. The resulting device was clear and exhibited a degree of flexibility.

The following details the method utilized to monitor timolol drug release from an ocular device of this example. Solutions of timolol maleate, in a concentration range of 5 ppm to 1,000 ppm, were prepared in Unisol® 4 buffer (Unisol® 4 is a preservative-free pH-balanced saline solution manufactured by Alcon Laboratories). A UV scanning spectrophotometer was utilized to generate a calibration curve of peak absorbance (λₘₐₓ=294 nm) versus concentration, in gm/100 ml, of timolol maleate. A calibration curve for timolol free base was then generated.

A device weighing between 100 and 150 mg was placed in a 4 ml vial. To the vial was added 2.0 ml of Unisol® 4 buffer. After 24 hours at 37° C., the sample was removed and placed in another 4 ml vial and covered with 2.0 ml of fresh Unisol® 4 buffer. The 24-hour release vial was capped, labeled and held for analysis. This procedure was repeated four more times to obtain 1-, 2-, 3-, 4- and 5-day release data. The sampling interval was then expanded to every 3 to 5 days and so on. The release study was carried out for a total of 90 days.

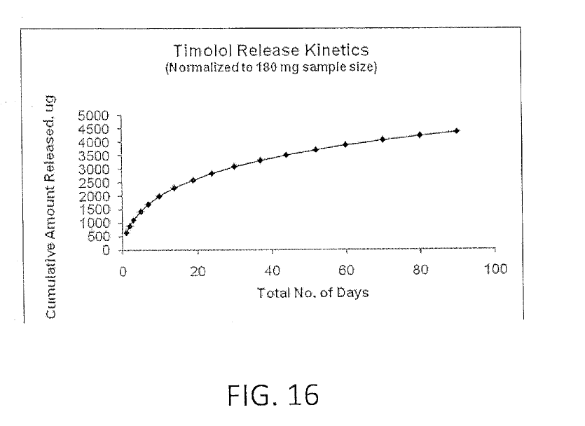

The drug release samples were analyzed by UV spectroscopy and absorbance readings converted to weight of drug via the calibration curve. A plot of cumulative weight, in micrograms, of drug released versus time was generated. The results were normalized to 0.180 gm of sample for convenience and are set forth in FIG. 16.

### Example 12

The following example describes a drug delivery system that is useful for the treatment of ocular infection and is based on a dispersed drug matrix. In this example the poorly (aqueous) soluble drug ciprofloxacin is dispersed in a hydrogel matrix. The following formulation, in two parts was prepared.

PART 1

| | | |
|---|---|---|
| Hydroxyethyl Methacrylate | 54.5 | ml |
| Polyethyleneglycol Dimethacrylate | 0.5 | ml |
| Ciprofloxacin Free Base | 5.0 | gm |
| | 60.0 | parts |

PART 2

| | | |
|---|---|---|
| Water | 50.0 | gm |
| 2,2-Azobis(2-methylpropionamide) | 0.375 | gm |
| dihydrochloride | | |
| | 50.375 | parts |

Mix above PART1 monomers then add the ciprofloxacin and utilize a Polytron Homogenizer for several minutes to micronize the drug and form a dispersion of ciprofloxacin. The following formulation was prepared:

| | |
|---|---|
| PART 1 | 1.5 ml |
| PART 2 | 1.0 ml |

The two piece polypropylene mold halves described in Examples 5 and 6 were utilized to produce devices of this invention. A prescribed amount of formulation is pipetted into the base half of the polypropylene mold. The second mold half, the cover, is fitted into the mold base to seal off the formulation and form the desired device geometry. The filled molds are placed in an oven and polymerized at 50° C. for 3 days.

The resulting devices then have an 8.33% (solids) loading of ciprofloxaein or based on 36.5% hydrated (equilibrium) water content the device has 5.3% ciprofloxacin in the hydrated.

The following details the method utilized to monitor ciprofloxacin drug release from an ocular device of this example. Solutions of ciprofloxacin, in a concentration range of 5 ppm to 1,000 ppm, were prepared in Purilens Plus buffer (Purilens Plus is a preservative-free pH-balanced saline solution manufactured by The Lifestyle Company). A UV scanning spectrophotometer was utilized to generate a calibration curve of peak absorbance (λₘₐₓ=272 nm) versus concentration, in gm/100 ml, of ciprofloxacin.

A device from this example weighing between 100 and 150 mg was placed in a 4 ml vial. To the vial was added 3.0 ml of Purilens Plus buffer. After 24 hours at 37° C., the sample was removed and placed in another 4 ml vial and covered with 3.0 ml of fresh Purilens Plus buffer. The 24-hour release vial was capped, labeled and held for analysis. This procedure was repeated four more times to obtain 1-, 2-, 3-, 4- and 5-day release data. The sampling interval was then expanded to every 3 to 5 days and so on. The release study was carried out for a total of 60 days.

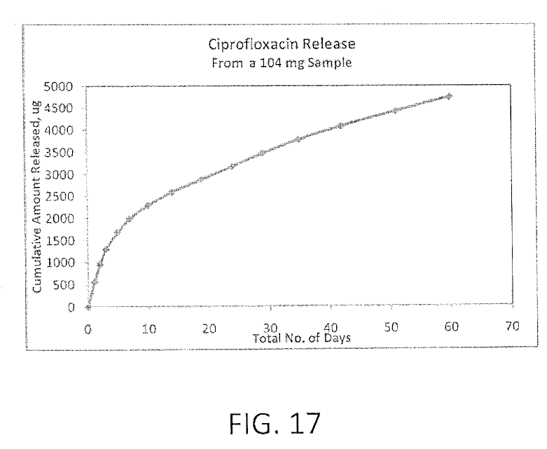

The drug release samples were analyzed by UV spectroscopy and absorbance readings converted to weight of drug via the calibration curve. A plot of cumulative weight, in micrograms, of drug released versus time was generated and is set forth in FIG. 17.

Based on the 60 days release study the amount of ciprofloxacin released was 4.74 mg compared to about an initial ciprofloxacin loading of 8.67 mg. This indicates that only 55% of the ciprofloxacin was released in the 60 days.

Examples 13 through 24 illustrate the many possible constructions and uses of the topical ocular drug delivery devices described in this invention. These examples should not be taken as limitations to the practice of this invention.

### Example 13

The devices of this invention can be in the form of at least one of the following: a matrix device with a dissolved therapeutic agent(s); a matrix device with a dispersed therapeutic agent(s); a matrix device with both a dispersed and dissolved therapeutic agents; a reservoir device with a solid therapeutic agents(s) core; a reservoir device with a liquid therapeutic agents(s) core; and a reservoir device with two internal cores of a different therapeutic agent(s)

### Example 14

The devices of this invention can be in the form of: a reservoir system wherein the reservoir contains a liquid to be delivered to the eye through a portal connecting the reservoir to the ocular environment. Said portal can be in the form of a small hole, valve, flap, screen or thin membrane. The liquid can then be directed to release over time to provide the eye with a therapeutic agent(s)

### Example 15

The devices of this invention can be in the form of: a device in the construction of an osmotic pump wherein the therapeutic agent(s) is released through a portal as a result of osmotic forces.

### Example 16

The devices of this invention can be in the form of: a reservoir or pump system wherein the therapeutic agent(s) is released through a portal(s), as a result of voluntary or involuntary contraction of the eyelid muscles or action of the blink.

### Example 17

The devices of this invention can be in the form of: a reservoir or pump system wherein the therapeutic agent(s) is released through a portal(s), as a result of voluntary or involuntary contraction of the extraocular muscles or action of eye movement.

### Example 18

The devices of this invention can be in the form of: a matrix system wherein the matrix is a hydrogel polymer containing from about 5% to 70% water and the therapeutic agent(s) is dissolved or dispersed uniformly in the hydrogel. In some cases it may be beneficial to include therapeutic agents that are both dissolved and dispersed in the same matrix.

### Example 19

The devices of this invention can be in the form of: a matrix system wherein the matrix is an erodible or biodegradable polymer or material and the therapeutic agent(s) is dissolved or dispersed uniformly in said matrix.

### Example 20

The devices of this invention can be in the form of: a non-degradable core with a coating that is erodible or biodegradable. The therapeutic agent(s) can be in either or both the core and the coating.

### Example 21

The devices of this invention can be in the form of: a combination device containing substantial components of non erodible material as well as erodible material. The therapeutic agent(s) can be in either, or both, materials.

### Example 22

The devices of this invention can be in the form of: a matrix system with dispersed nano-particles and/or micro-particles, or nano-spheres and/or microspheres, said particles containing a therapeutic agent(s)

### Example 23

The devices of this invention can be in the form of: a matrix or reservoir system wherein the “top”, or distal, surface of the device is coated with a barrier material to prevent the release of therapeutic agent(s) through this surface. Thus the release of all the therapeutic agent(s) will be through the “bottom”, or proximal, surface that is in contact with the conjunctiva or sclera.

### Example 24

The devices of this invention can be in the form of: a device containing one or more cavities or “holes” that contain therapeutic agents(s) to be released directly into the eye. Also a combination of a matrix system containing a dissolved or dispersed therapeutic agent(s) with cavities or “holes’ containing other therapeutic agent(s).

All of the designs, compositions, constructions and methods disclosed and claimed herein can be made and executed without undue experimentation in light of the present disclosure. While, the designs and methods of this invention have been described in terms of preferred embodiments, it will be apparent to those skill in the art that variations may be applied to the designs and/or methods and in the steps or in the sequence of steps of the methods described herein without departing from the concept, spirit and scope of the invention. All such similar substitutes and modifications apparent to those skilled in the art are deemed to be within the spirit, scope and concept of the invention as described by the appended claims.

1. An topical ocular system for delivery of a therapeutic agent to an eye comprising:
a body having an anterior surface and a posterior surface for placement on one of a superior sclera and inferior sclera of the eye, wherein the posterior surface is defined by a base curve and edge lift radii at peripheral edges thereof that are both complementary to and adapted to fit the sclera of the eye so as to permit the device to the held on the eye by fluid attraction and be retained on the eye without aid of an eyelid, where said ocular device matrix composed of a polymeric material, or a combination of polymeric materials, said ocular device containing a therapeutic agent, or a combination of therapeutic agents, said ocular device releasing the therapeutic agent or agents in a controlled manner, the mechanism of release is either physical/physiochemical or chemical/biochemical or a combination of the above two mechanisms.

2. An ocular system of claim 1, wherein the polymeric matrix comprises a non-erodible polymer or an erodible polymer or a combination of both.

3. An ocular system of claim 1, wherein the therapeutic agent, or a combination of therapeutic agents is dissolved in the polymeric matrix.

4. An ocular system of claim 1, wherein the therapeutic agent or a combination of therapeutic agents is dispersed in the polymeric matrix.

5. An ocular system of claim 1, wherein the therapeutic agent or a combination of therapeutic agents is contained in a reservoir either totally internal to the polymer matrix or partially exposed to the ocular environment.

6. An ocular system of claim 5, wherein the therapeutic agent or a combination of therapeutic agents is contained in a reservoir either totally internal to the polymer matrix or partially exposed to the ocular environment. The therapeutic agent or a combination of therapeutic agents forming the reservoir are either neat or admixed with a polymer.

7. An ocular system of claim 1, wherein the therapeutic agent or a combination of therapeutic agents are selected from the group consisting of: analgesics, mydriatics and mydriolytics, antiglaucoma drugs, anti-infective drugs, anti-inflammatory drugs, antiallergy drugs, antiangiogenic drugs and lubricants.

8. An ocular system of claim 1, wherein the drug containing device is placed underneath the conjunctiva or Tenon's capsule for drug delivery to the anterior segment and/or posterior segment of the eye.

9. An ocular system of claim 8, wherein the ocular diseases to be treated include: glaucoma, age related macular degeneration, diabetic macular edema, uveitis, and retinitis.

10. An ocular system of claim 1, wherein the therapeutic agent or a combination of therapeutic agents is a prostaglandin alone or in combination with another ocular drug.

11. An ocular system of claim 10, wherein the prostaglandin is dispersed throughout the polymeric matrix or localized in a portion of the device.

12. An ocular system of claim 11, wherein the prostaglandin is localized in a cavity that has an exposed surface on the base curve of the device.

13. An ocular system of claim 12, wherein the prostaglandin is dispersed in a polysiloxane and is localized in a cavity that has an exposed surface on the base curve of the device.

14. An ocular system in accordance with claim 6, wherein the ocular device contains both a prostaglandin in a cavity or a prostaglandin dispersed in a polysiloxane in a cavity and another anti-glaucoma agent that is dissolved or dispersed throughout the device matrix.
